(19) United States
(12) Patent Application Publication
(10) Pub. No.: US 2011/0172767 A1
(43) Pub. Date: Jul. 14, 2011

(54) MINIMALLY INVASIVE, DIRECT DELIVERY METHODS FOR IMPLANTING OBESITY TREATMENT DEVICES

(76) Inventors: Pankaj Rathi, (US); Theodore M. Bender, (US); Brian K. Shiu, (US); Pablo G. Acosta, (US); Joshua Makower, (US); Shuji Uemura, (US); Narvel M. Brooks, III, (US); Robert M. George, (US); Earl A. Bright, II, (US); Dane A. Johnson, (US); Matthew B. Newell, (US); Marlo Dreissigacker, (US); Crystine M. Lee, (US)

(21) Appl. No.: 13/015,086

(22) Filed: Jan. 27, 2011

Related U.S. Application Data
(63) Continuation-in-part of application No. 12/474,226, filed on May 28, 2009.
Application No. 12/474,226 is a continuation-in-part of application No. 11/716,985, filed on Mar. 10, 2007.
Application No. 11/716,985 is a continuation-in-part of application No. 11/716,986, filed on Mar. 10, 2007.
Application No. 11/716,986 is a continuation-in-part of application No. 12/473,818, filed on May 28, 2009.
Application No. 12/473,818 is a continuation-in-part of application No. 11/716,985, filed on Mar. 10, 2007.
Application No. 11/716,985 is a continuation-in-part of application No. 11/716,986, filed on Mar. 10, 2007.
Application No. 11/716,986 is a continuation-in-part of application No. 11/474,118, filed on Jun. 22, 2006, now abandoned.
Application No. 11/474,118 is a continuation-in-part of application No. 11/407,701, filed on Apr. 19, 2006.
Application No. 11/407,701 is a continuation-in-part of application No. 11/974,444, filed on Oct. 11, 2007.

(51) Int. Cl. A61F 2/02; A61B 17/10; A61M 29/00; B32B 37/02; B32B 38/00; B32B 37/06

(52) U.S. Cl. 623/11.11; 606/139; 606/191; 156/60; 156/293

(57) ABSTRACT

A method includes selecting a template from a plurality of different sizes of templates based on measurements of the abdominal cavity of a patient; orienting the template on the patient at a location overlying the abdominal cavity to select an appropriate size implant using fluoroscopic imaging; marking an incision location and an indicator of an angle of approach; and removing the template from the patient, wherein marks made by the marking remain on the patient. Methods apparatus, instruments and implants for treating a patient are provided.

### CROSS-REFERENCE

This application is a continuation-in-part application of co-pending application Ser. No. 12/474,226, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 12/473,818, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 12/474,118, filed May 28, 2009, which is a continuation-in-part application of application Ser. No. 11/716,985, filed Mar. 10, 2007 and a continuation-in-part application of application Ser. No. 11/716,986, filed Mar. 10, 2007, and we hereby claim priority to each of the foregoing applications under 35 USC §120. Each of the foregoing applications is also hereby incorporated herein, in its entirety, by reference thereto.

This application is a continuation-in-part application of co-pending application Ser. No. 11/407,701, filed Apr. 19, 2006 to which application we claim priority under 35 USC §120 and which application is incorporated herein, in its entirety, by reference thereto.

This application is a continuation-in-part application of co-pending application Ser. No. 11/974,444, filed Oct. 11, 2007 to which application we claim priority under 35 USC §120 and which application is incorporated herein, in its entirety, by reference thereto.

### FIELD OF THE INVENTION

The present invention relates to the field of minimally invasive surgery, and more particularly to methods, devices, tools and systems for abdominal surgical procedures employing an endoscope for at least part of a procedure.

### BACKGROUND OF THE INVENTION

There is a current ongoing trend toward the advancement of minimally invasive surgical techniques. Such techniques not only reduce the amount of trauma to the patient, but consequently reduce the amount of recovery time needed for healing, thereby reducing the lengths of hospital stays and, in some cases, even making it possible to perform procedures on an outpatient basis, such as in a physician's office.

Examples of existing procedures include laparoscopic procedures, wherein a procedure is conducted transdermally to reach an internal surgical target location. Typically this involves the formation of several (typically three or more) ports or openings through the skin and into the patient, for placement of an endoscope through one opening and tools, instruments, devices through the other openings.

Other examples of existing procedures include those where an endoscope and/or other instrumentation is inserted through a natural orifice, such as the mouth, anus, vagina, etc. The endoscope/instrument may be advanced along a natural pathway and then used to access the surgical site by piercing through a natural conduit forming the natural pathway. Alternatively, a procedure may be performed within the natural pathway, or on the natural conduit forming the natural pathway.

In any of these cases, the use of an endoscope may be limited when obstacles are present in a pathway leading to the surgical target location. Such obstacles may be fat or other soft tissue obstruction, tumors, or even the fact that the route from the insertion location of the endoscope/instrument to the surgical target location is very tortuous, making it difficult to establish a pathway to the surgical target location.

Traditionally, suturing has been performed to attach devices to tissues, to attach tissues to one another and/or to close wounds and incisions. However, successful suturing requires significant skill to perform, is time consuming, and is often difficult, if not impossible to perform in a minimally invasive procedure through a port, or even through multiple ports in a laparoscopic procedure.

Alternatives to suturing are known, but may result in less desirable outcomes. For example, gastric reduction techniques have been attempted, such as by inserting instruments trans-orally and reducing the volume of the stomach by stapling portions of it together. However, this technique is prone to failure due to the staples pulling through the tissues that they are meant to bind.

In an example of laparaoscopic hernia repair, multiple instruments are used through multiple ports to conduct the repair, but suturing is often replaced by stapling due to the reduced access space that is not sufficient to successfully carry out the suturing operations.

It would be desirable to provide instruments and techniques useable in less invasive surgical methods, such as minimally invasive surgical procedures using only one small opening into a patient, or laparaoscopic surgical procedures using two to five small openings into the patient, that provide the capability of fastening by sutures to fasten a device to an anatomical structure, to repair an opening or tear, or to otherwise fasten two or more tissues together.

### SUMMARY OF THE INVENTION

The present invention provides methods, apparatus, instruments and/or implants for treating a patient.

In one aspect of the present invention, a method is provided that includes: selecting a template from a plurality of different sizes of templates based on measurements of the abdominal cavity of a patient; orienting the template on the patient at a location overlying the abdominal cavity to select an appropriate size implant; marking an incision location and an indicator of an angle of approach; and removing the template from the patient, wherein marks made by the marking remain on the patient.

In at least one embodiment, the method includes using fluoroscopic imaging to facilitate selection of the appropriate size implant.

In at least one embodiment, the method includes selecting the appropriate size implant from a plurality of different sizes of implants.

In at least one embodiment, the implants are enlargeable implants.

In at least one embodiment, the method includes marking the patient at a location overlying a portion of the costal margin, prior to the orienting, and wherein the orienting includes positioning a superior edge of a cutout in the template adjacent to or inferior of a mark made by the marking the location overlying a portion of the costal margin.

In at least one embodiment, the method includes adhering a marking guide to an indicator location as the indicator of the angle of approach.

In at least one embodiment, the template includes a cutout indicating a location where the implant is to be attached to the abdominal wall, and wherein the indicator of the angle of approach comprises a mark drawn within bounds of the cutout.

In at least one embodiment, the method includes adhering a marking guide to the patient so that the marking guide overlies the mark drawn within bounds of the cutout.

In at least one embodiment, the method includes positioning the template so that a head of the template approximates the diaphragm of the patient, but does not extend superiorly of the diaphragm.

In at least one embodiment, the method of marking the incision location comprises marking adjacent to a notch in a tail of the template or inferior of the notch, adjacent to a portion of the tail inferior of the notch.

In at least one embodiment, the method includes adhering a marking guide to a location overlying a portion of the costal margin.

In at least one embodiment, the method includes adhering a marking guide to the patient, wherein the marking guide overlies a mark made by the marking of the patient at a location overlying a portion of the costal margin.

In at least one embodiment, the method includes placing a suture marker that extends along the internal surface of the abdominal wall along the inferior edge of a portion of the costal margin of the patient.

In at least one embodiment, the method includes: making an incision or puncture through the patient's skin at the marked incision location; establishing a delivery tract through an opening formed by the incision or puncture, subcutaneous fat and fascia and into the patient's abdominal cavity, but not through the stomach; dilating the opening and placing an introducer cannula along the tract such that the introducer cannula extends from a location outside of the patient to a location within the abdominal cavity; inserting an instrument and the selected enlargeable implant into the introducer cannula, wherein the enlargeable implant is mounted on a distal end portion of the instrument and the enlargeable implant is in a compact configuration; enlarging the implant to an enlarged configuration; attaching the implant to an inner surface of the abdominal cavity; removing the instrument and introducer cannula; attaching an adjustment member to a fill tube in fluid communication with the implant; and closing the opening.

In at least one embodiment, the method includes, prior to enlarging the implant, retracting the introducer cannula relative to the instrument and implant to expose the implant and a working end of the instrument out of a distal end of the introducer cannula.

In at least one embodiment, the method includes, prior to attaching the implant, contacting a lowermost rib of the patient with a depression formed in a distal end portion of the instrument extending distally of an end effector of the instrument that is configured to drive stitches.

In at least one embodiment, the method includes attaching a sealing member mounted on the instrument to a proximal end of the introducer cannula to seal off the introducer cannula; and insufflating the abdominal cavity of the patient.

In at least one embodiment, the method includes prior to the attaching the implant, verifying a correct positioning of the implant by verifying alignment of the instrument with the indicator of the angle of approach.

In at least one embodiment, the method includes, prior to attaching the implant, verifying a correct positioning of the implant by verifying alignment of the instrument with at least one of the indicator of the angle of approach and the suture marker.

In at least one embodiment, the method includes, prior to attaching, verifying a position of a working end of the instrument relative to the marking guide that overlies a portion of the costal margin.

In at least one embodiment, the method includes verifying, using direct laparoscopic visualization, a location of a distal end of an end effector of the instrument relative to the costal margin of the patient.

In at least one embodiment, the dilation of the opening and placement of the introducer cannula comprises inserting a distal end of a dilator through the opening, wherein the introducer cannula is mounted over the dilator and a distal end portion of the introducer cannula is passed through the abdominal wall along the tract, and the method further includes removing, the dilator prior to the inserting an instrument and enlargeable implant into the introducer cannula; and insufflating the abdominal cavity.

In at least one embodiment, the opening is the only opening formed in the patient to carry out the entirety of the method.

In at least one embodiment, the implant is attached to at least one of fascia, peritoneum, preperitoneal fat and/or posterior rectus sheath.

In at least one embodiment, the implant is attached to abdominal muscle.

In at least one embodiment, the instrument comprises an attachment tool and a suturing tool that are releasably connected to one another, wherein the attaching comprises attaching the implant using the attachment tool, and then disconnecting the attachment tool from the suturing tool and removing the attachment tool from the introducer cannula.

In at least one embodiment, the method includes tightening the attachment of the implant to the inner surface of the abdominal cavity using the suturing tool; and wherein the removing of the instrument comprises removing the suturing tool after completing the tightening of the attachment.

In at least one embodiment, the method includes removing at least a portion of a falciform ligament.

In another aspect of the present invention, an apparatus for use in a minimally-invasive abdominal surgical procedure is provided that includes: an elongate introducer cannula having a tubular main body, a distal end, a proximal end and a main lumen extending therethrough; a stitching instrument having at least one elongate shaft insertable through the introducer cannula, the instrument having a length greater than a length of the elongate introducer; a sealing member forming a seal around the at least one elongate shaft of the instrument and configured to form a seal between the instrument and the introducer cannula to seal off the main lumen; and an enlargeable implant releasably attached to a distal end portion of the instrument.

In at least one embodiment, the stitching instrument comprises an attachment tool and a suturing tool that are releasably connected to one another, wherein the attaching comprises an end effector having needles configured to drive stitches to attach the implant to a patient.

In another aspect of the present invention, a sealing member for forming a seal between an introducer cannula and an attachment tool configured to attach an implant in the abdominal cavity is provided, the sealing member including: a main body having a generally circular cross-sectional configuration; attachment members configured and dimensioned to attach to a proximal end of the introducer cannula; a sealing ring configured to seal with an opening in fluid communication with a main lumen of the introducer cannula; an opening configured to allow passage of an end effector having a first cross-sectional area, as well as a shaft having a second cross-sectional area different from said first cross-sectional area; and a valve formed around the opening and configured to form a seal with the shaft and the opening.

In at least one embodiment, the opening comprises a first opening and the valve comprises a first valve, the sealing member comprising a second opening for receiving a tool or instrument therethrough and forming a seal therewith, and a second sealing member formed around the second opening.

In another aspect of the present invention, an implantable device for treatment of obesity is provided, the device including: an expandable main body member configured to be positioned adjacent a portion of a stomach of a patient, within the abdominal cavity of the patient wherein the expandable main body member comprises a wall surrounding an internal chamber; an attachment tab interfacing with an outer surface of the wall and extending from the wall of the expandable main body member, the attachment tab configured to fix a portion of the main body member to and in contact with a portion of at least one internal body structure; an inner backing layer interfacing with an inner surface of the wall and bonded thereto; wherein the attachment tab, the wall and the inner backing layer are bonded together.

In at least one embodiment, the attachment tab and the inner backing layer are bonded together through at least one opening through the wall and each opening is sealed by the bonding together of the attachment tab and the inner backing layer.

In at least one embodiment, the device further includes a plug bonded to the attachment tab, the wall and the inner backing layer, the plug having placed in an opening in the wall, the plug having been bonded with the attachment tab, wall and inner backing layer, thereby filling the opening in which the plug was inserted prior to having been bonded.

In another aspect of the present invention, a method of making an implantable device for treatment of obesity is provided, the method including: providing an expandable main body member configured to be positioned adjacent a portion of a stomach of a patient, within the abdominal cavity of the patient wherein the expandable main body member comprises a wall surrounding an internal chamber and an opening through the wall; laying a layer of an attachment tab on an outer surface of the wall over a location of the opening; contacting an inner backing layer to an inner surface of the wall under a location of the opening; and bonding the wall, layer of an attachment tab and inner backing layer together.

In at least one embodiment, the method further includes inserting a plug in the opening, and the bonding comprises bonding the wall, plug layer of an attachment tab and inner backing layer together.

In at least one embodiment, the bonding comprises vulcanizing.

These and other advantages and features of the invention will become apparent to those persons skilled in the art upon reading the details of the methods, apparatus, instruments and implants as more fully described below.

### BRIEF DESCRIPTION OF THE DRAWINGS

FIG. 1 illustrates an embodiment of a surgical apparatus that is configured to deliver an implantable device, assembled thereon, from outside of a patient, through an opening and into the patient.

FIGS. 2A-2B show a proximal end perspective view and a distal end perspective view of one embodiment of a sealing member according to the present invention.

FIG. 2C illustrates the sealing member of FIGS. 2A-2B having been installed on an apparatus according to one embodiment of the present invention.

FIG. 2D illustrates the working end portions of the apparatus of FIG. 2C having been inserted into an introducer cannula, and the sealing member of FIGS. 2A-2C having been attached to the proximal end of the introducer cannula.

FIG. 2E illustrates the capability of axially advancing the apparatus of FIG. 2C relative to the introducer cannula to extend the working ends of the apparatus distally of the distal end of the introducer cannula while maintaining the seal between the proximal end of the introducer cannula and the shafts of the assembly, according to an embodiment of the present invention.

FIG. 2F illustrates the capability of an elastic valve of the sealing member of FIGS. 2A-2E to expand while still conforming to the cross-sectional shape of the portion of the instrument extending therethrough, according to an embodiment of the present invention.

FIG. 2G illustrates use of a closure member to positively seal off the elastic valve of FIG. 2F after complete removal of the tool therefrom.

FIGS. 3A-3B show a proximal end perspective view with the first valve in a closed configuration, and with the first valve in an open configuration, respectively, of another embodiment of a sealing member according to the present invention.

FIG. 3C shows a distal end perspective view of the sealing member of FIGS. 3A-3B.

FIG. 3D illustrates the sealing member of FIGS. 3A-3C having been installed on an apparatus according to one embodiment of the present invention.

FIG. 3E illustrates the working end portions of the apparatus of FIG. 3D having been inserted into an introducer cannula, and the sealing member of FIGS. 3A-3D having been attached to the proximal end of the introducer cannula, according to an embodiment of the present invention.

FIG. 3F illustrates the capability of axially advancing the apparatus of FIG. 3D relative to the introducer cannula to extend the working ends of the apparatus distally of the distal end of the introducer cannula while maintaining the seal between the proximal end of the introducer cannula and the shafts of the assembly, according to an embodiment of the present invention.

FIG. 3G illustrates the capability of a first valve of the sealing member of FIGS. 3A-2F to expand while still conforming to the cross-sectional shape of the portion of the instrument extending therethrough, according to an embodiment of the present invention.

FIG. 3H illustrates the first valve in a closed configuration to positively seal off the port that it is formed around, after complete removal of the tool therefrom.

FIGS. 4A-4B show a proximal end perspective view of another embodiment of a sealing member, with the first valve in a closed configuration, and with the first valve in an open configuration, respectively.

FIG. 4C shows a distal end perspective view of the sealing member of FIGS. 4A-4B.

FIG. 4D illustrates the sealing member of FIGS. 4A-4C having been installed on an apparatus according to one embodiment of the present invention.

FIG. 4E illustrates the working end portions of the apparatus of FIG. 4D having been inserted into an introducer cannula, and the sealing member of FIGS. 4A-4D having been attached to the proximal end of the introducer cannula, according to an embodiment of the present invention.

FIG. 4F illustrates the capability of axially advancing the apparatus of FIG. 4D relative to the introducer cannula to extend the working ends of the apparatus distally of the distal end of the introducer cannula while maintaining the seal between the proximal end of the introducer cannula and the shafts of the assembly, according to an embodiment of the present invention.

FIG. 4G illustrates the capability of a first valve of the sealing member of FIGS. 4A-4F to expand to allow withdrawal of the working end portion of a tool that has a larger cross-sectional area than a shaft thereof.

FIG. 4H illustrates the ability of the first valve shown in FIG. 4G to close down to a smaller configuration after removal of the working end.

FIGS. 5A-5B show a proximal end perspective view and a distal end view of a sealing member, respectively, according to an embodiment of the present invention.

FIG. 6A shows a plug that is insertable into a port of a sealing member according to an embodiment of the present invention.

FIG. 6B shows another plug that is insertable into another port of the sealing member according to an embodiment of the present invention.

FIG. 7 illustrates a dilator according to an embodiment of the present invention.

FIG. 8 illustrates an introducer cannula according to an embodiment of the present invention.

FIGS. 9A-9D illustrate an introducer/cannula that is insertable into a patient in a first configuration and then is expandable to a second expanded configuration, according to an embodiment of the present invention.

FIG. 10 is a partial illustration of a shaft of an introducer/cannula according to another embodiment of the present invention.

FIG. 11 illustrates an implantable device according to an embodiment of the present invention, configured for delivery and paragastric, extragastric implantation.

FIG. 12A is an exploded view of an attachment tab with an alternative layup arrangement for bonding the attachment tab to the expandable member 10em, according to an embodiment of the present invention.

FIG. 12B illustrates an end view of the attachment tab of FIG. 12A having been bonded to the expandable member 10em.

FIG. 12C is a longitudinal sectional view taken from FIG. 12B.

FIG. 12D is a detailed view of the vulcanized joint indicated within circle 12D of FIG. 12C.

FIG. 13A is a partial view of one embodiment an endoscope that may be used in procedures described herein according to the present invention.

FIG. 13B shows a longitudinal sectional view of the endoscope in FIG. 11A.

FIGS. 14A-14N illustrate an example of a procedure for directly implanting an extra-gastric device according to an embodiment of the present invention.

FIGS. 15A-15T illustrate an example of a procedure for directly implanting an extra-gastric device according to another embodiment of the present invention.

FIGS. 16A-16F illustrate events during the preparation of an instrument assembly and enlargeable implant for use according to an embodiment of the present invention.

FIGS. 17A-17O illustrate events carried out during template size selection and location device size selection according to an embodiment of the present invention.

FIGS. 18A-18C illustrate placement and use of an optional suture marker according to an embodiment of the present invention.

FIG. 19 illustrate a distal end portion of a suturing instrument according to another embodiment of the present invention.

### DETAILED DESCRIPTION OF THE INVENTION

Before the present apparatus, devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a tool” includes a plurality of tools and reference to “the suture” includes reference to one or more sutures and equivalents thereof known to those of ordinary skill in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A “proximal” end of an instrument is the end that is nearer the surgeon when the surgeon is using the instrument for its intended surgical application.

A “distal” end of an instrument is the end that is further from the surgeon when the surgeon is using the instrument for its intended surgical application.

An “internal body structure” refers to a structure internal to the skin of a patient, and which can be within the abdominal cavity or other cavity of the patient, or just outside of it, such as including the outer surface of a wall that partially defines the cavity. Further, an internal body structure may be located anywhere in the body internal to the skin.

A “surgical target location” or “surgical target area” as used herein refers to a location internal of a patient where a surgical procedure is to be performed. Such surgical procedures include, but are not limited to, treatment of existing tissues with one or more tools and/or implantation of one or more devices at the surgical target location.

### Tools, Devices, Systems and Methods

The preferred embodiments of the present invention facilitate minimally-invasive procedures for implanting one or more devices within a patient, and/or minimally invasive features for joining tissues or repairing tissue defects such as a hernia, for example.

Thus, although the majority of the specific embodiments focus on implantation of a device to treat obesity, the present tools and methods are not limited to such procedures, as tools described herein may be used in other minimally invasive procedures, including, but not limited to hernia repair.

Preferred embodiments include use of an attachment tool that is useable from a location outside of a patient to attach a device internally to a patient or to perform repairs of tissue defects, etc. Advantageously, apparatus provided are configured to and capable of applying sutures to a target arranged substantially in a flat plane or having a slightly curved surface. Thus tissue does not have to be sucked in, folded, bunched up, or otherwise gathered in order to apply sutures as is required for prior art tools.

In at least one procedural embodiment, a tract is established from an opening in a patient that opens to the outside of the patient, to a surgical target location located internally of the patient. Direct visualization through a preferred device is possible during the establishment of such tract.

In preferred embodiments, a minimally-invasive procedure includes use of insufflation of the abdominal cavity during performance of one or more procedural steps performed. This application of insufflation allows the procedure to use a fewer number of tools relative to the procedures described in the parent applications that use no or only minimal amounts (e.g., “a puff” or about 0.5 liters or less of carbon dioxide) of insufflation. Preferably, only a single small opening is required for insertion of the tools/devices and optionally, an implantable device. The small opening will generally be less than about 2.5″ in diameter, or less than about 2.2″ in diameter, or less than about 2″ in diameter, or less than about 1.5″, less than about 1.25″ or less. For use with general anesthesia, the opening may be up to about 3 inches in diameter or up to about 3.5 inches in diameter. Alternatively, more than one opening may be used for viewing through and/or inserting additional instruments.

For weight loss applications, weight loss is achieved by restriction of the stomach and filling of the space into which the stomach normally expands into the abdominal cavity when filled with food. An implantable device expands outwardly when filled to occupy space within the abdominal cavity such that when food is ingested the stomach is restricted from being able to hold any more than a small volume of food. The implantable, outwardly expandable device is implanted outside of the stomach in the left upper quadrant of the abdominal cavity to achieve these functions. The expandable portion of the implantable device does not pierce or encircle nerve tissue or other tissue. The implantable, expandable device may be positioned with direct visualization (i.e., using an endoscope) and/or fluoroscopic visualization. No dissection, suturing, attachment or other invasive manipulation or trauma into or on the stomach is required in order to implant the implantable, expandable device. By appropriate placement of the implantable, expandable device, the device can achieve restriction of the stomach. Further, the volume of the implantable, expandable device is adjustable so that the amount of restriction of the stomach can be adjusted. This can be advantageous over time, as the patient may be able to accept, or require, additional restriction of the stomach as weight loss progresses. Likewise, the loss of fat in the abdominal cavity may require the implantable, expandable device to be increased in volume to occupy additional space that is freed up by the weight loss. Both the shape of the implantable, expandable device and its fill volume, in combination, cause the desired stomach compression. Implant materials are chosen that are compatible with magnetic resonance imaging (MRI), computed tomography (CT) imaging, fluoroscopy, and X-ray imaging.

Implantation of the implantable, outwardly expandable device is carried out so as not to encircle any muscle or nerve tissue with the expandable member. Various implantable, outwardly expandable device sizes are provided, so that the present invention can treat a wide range of patients, with BMI's ranging from about 35 to about 50 and above, and including different rib cage dimensions. The present invention minimizes stress to the stomach.

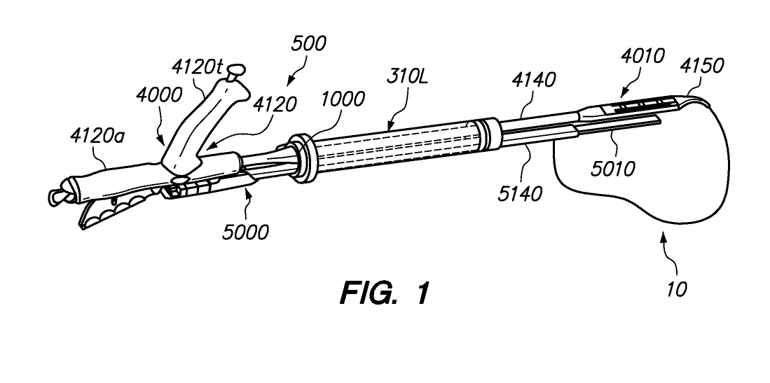

FIG. 1 illustrates an embodiment of an implantable device 10 (shown in an enlarged or expanded configuration) assembled on a surgical apparatus 500 that is configured to deliver the device 10 from outside of a patient, through an opening and into the patient (e.g., into the abdominal cavity of the patient), and to implant the device 10 by suturing it to a surgical target location within the patient, e.g., the internal wall surface of the abdominal cavity, internal fascia, and/or some other internal body structure. Implant device 10 is inserted into the patient in a compact, non-expanded configuration. Apparatus 500 includes a stitching instrument 4000 releasably coupled with a suturing instrument 5000. Stitching instrument 4000 includes a working end portion 4010 that is preferably radiolucent so that the needles and suture anchors are easier to visualize when using fluoroscopy, with the working end portion 4010 having been inserted into the patient. Working end portion 4010 is provided at a distal end portion of the instrument from which and into which end effectors (e.g., tissue pins, stitching needles) move, as described in more detail in co-pending U.S. application Ser. Nos. 12/474,226; 12/473,818; and 12/474,118, which were incorporated herein above, in their entireties, by reference thereto. An elongate shaft 4140 extends between working end portion 4010 and handle 4120. In one embodiment, shaft 4140 has a length from the distal end of handle 4120 to the proximal end of working end portion 4010 of about 20.25″±about 0.25″, where the overall length of the instrument 400 is about 37.2″ (excluding the length of guide 4150). With the implant guide 4150, the overall length is about 40″. All of the foregoing length measurements may vary depending on multiple factors including, but not limited to: the size of the implant 10 to be delivered, the size of the patient, etc. Shaft 4140 has a length sufficient to allow a user to operate the controls on handle 4120 from a location outside of an obese or overweight patient when the working end portion 4010 is contacted to a surgical target area where stitching and suturing are to be performed. Handle 4120 includes an axial portion 4120a and a transverse portion 4120t. These portions are configured so that the user can apply both hands to the handle 4120 if desired and, by pulling on handle portion 4120t and pushing down on handle portion 4120a can apply a force to the working end portion 4010 to press it up against a surgical target where stitching and suturing are to be performed.

The apparatus shown in FIG. 1 is substantially the same as that used in previous methods described in one or more of the applications that have been incorporated herein where no or only minimal amounts of insufflation are performed. However, the apparatus in FIG. 1 differs in that a sealing member 1000 is provided abound the shafts 4140 and 5140 of the apparatus 500. Sealing member 1000 may be configured to function as an end plug to seal off the space between a tubular member and the shafts 4140 and 5140, as illustrated in FIG. 1. Alternatively, sealing member 1000 may be configured as a sliding plug that slides within the annulus of a tubular member to seal off the space between the tubular member and one or more tools or shafts, an example of which is described in more detail below.

In the embodiment of FIG. 1, sealing member 1000 is shown attached to a proximal end of a large cannula or introducer 310L to seal off the proximal opening of the introducer 310L by sealing off the space between shafts 4140, 5140 and the inner wall of introducer 310L. In an alternative embodiment, the sealing member 1000 can be used in like manner to seal off a proximal end of a conduit used for delivery of device 10.

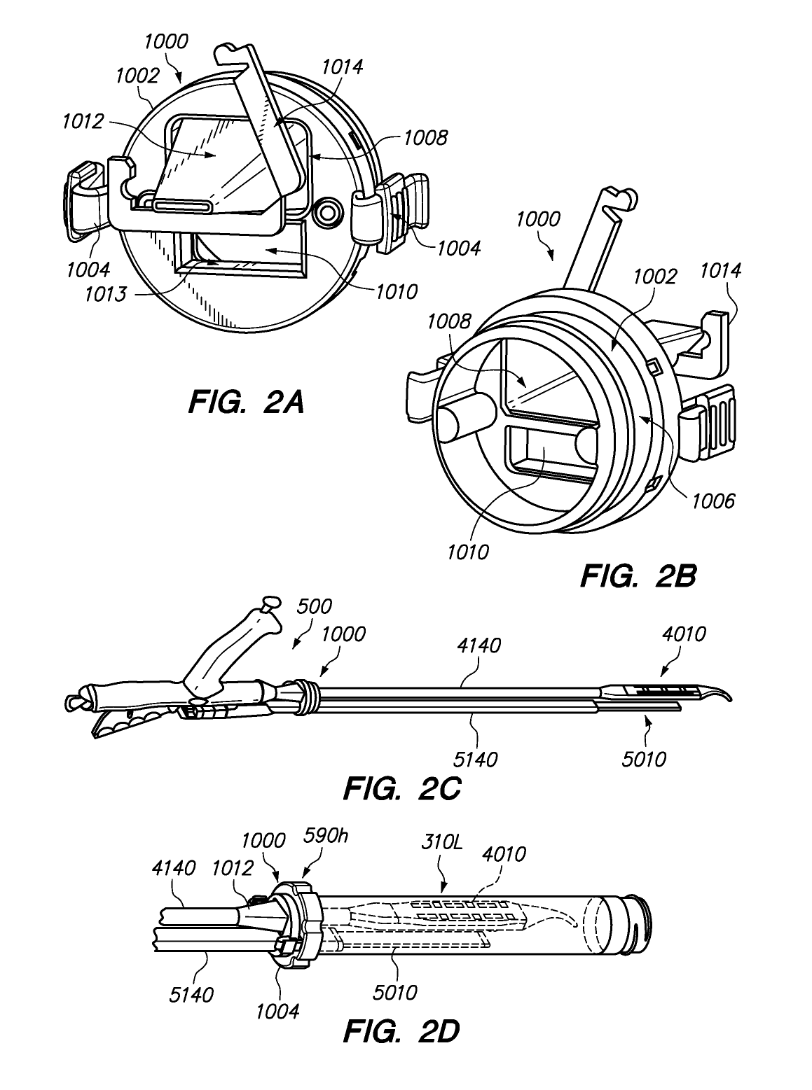

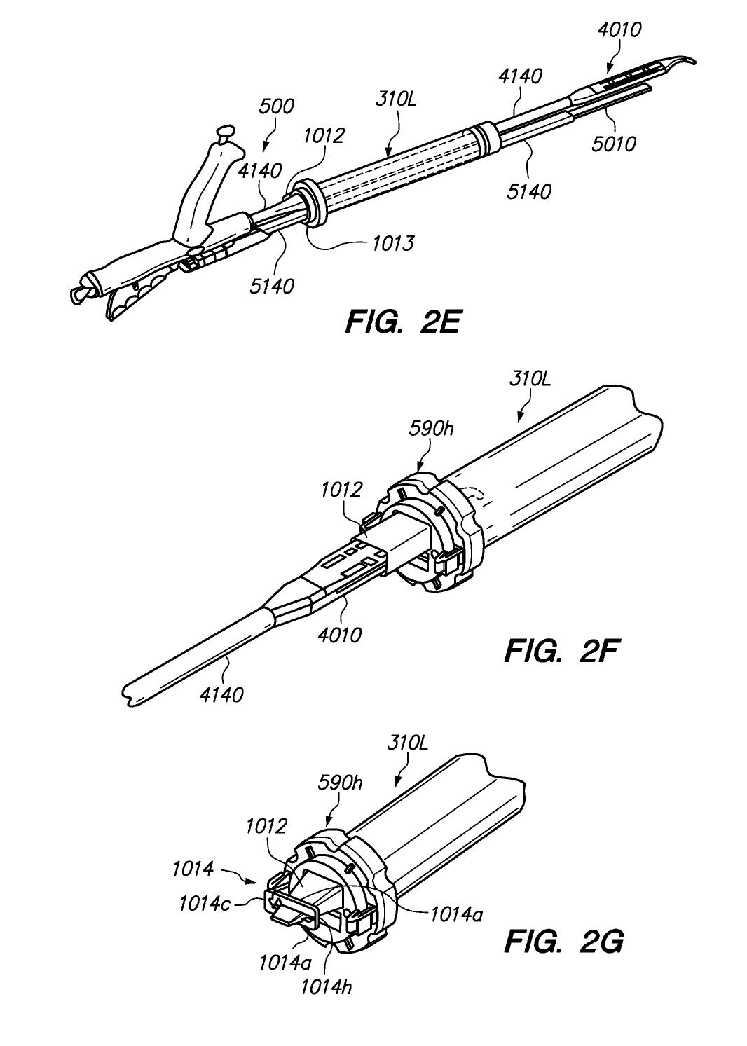

FIGS. 2A-2B show a proximal end perspective view and a distal end perspective view of a sealing member 1000 according to an embodiment of the present invention. Sealing member 1000 is configured and dimensioned to form a seal between a tubular member such as introducer cannula 310L and apparatus 500 and/or one or more other surgical tools or instruments. Sealing member 1000 includes a main body 1002 having a generally circular cross-sectional configuration. Attachment members 1004 are provided to facilitate attachment of the sealing member 1000 to introducer cannula 310L or other tubular member where a seal is to be formed. As shown, attachment members 1004 are clips that form a snap fit with the distal end of the tubular member or an end cap thereof.

A sealing ring 1006 (such as an O-ring or the like) is provided on the distal end portion of main body 1000. the distal end portion of the main body 1000 is configured to slide within the lumen of the introducer cannula 310L with a close fit and sealing ring 1006 forms a friction fit with the inner wall of the introducer cannula 310L, thereby forming a seal between the sealing member body 1000 and the inner wall of the introducer cannula.

Main body 1000 is provided with two ports: a first port 1008 configured and dimensioned to receive the working end 4010 and shaft 4140 of attachment tool 4000 therethrough; and a second port 1010 configured and dimensioned to receive the working end 5010 and shaft 5140 of suturing tool 5000 therethrough. Because shaft 4140 has a different cross-sectional shape than working end 4010, an elastic valve is sealed around the perimeter of first port 1008 and extends therefrom to provide a sealing valve having variable cross-sectional shapes and dimensions. In the embodiment of FIG. 2A, the first elastic valve 1012 is a duckbill vale shaped to look substantially like a duck's bill. That is, elastic valve 1012 tapers from a largest cross-sectional, rectangular shape and dimension to a smallest cross-sectional dimension, which may be rectangular or an elongated slit. The taper progressively reduces in cross-sectional dimension in a direction form the largest end to the end having the smallest cross-sectional dimension. Thus, elastic valve 1012 functions like a sock or glove to conform to the cross sectional dimension of the instrument or portion of an instrument extending through the opening thereof and through the valve. As shown, elastic valve 1012 extends proximally of the proximal end of main body 1000. Although elastic valve 1012 could extend distally of the proximal end of main body 1000, the configuration shown in FIG. 2A allows for use of a closure member 1014 such as snap clip 1014 to completely close off and seal the opening into the valve 1012 when no instrument is inserted therethrough. Additionally, or alternatively, elastic valve 1012 can be configured to automatically seal off when no instrument is inserted therethrough. An elastic seal 1013 is formed around the perimeter of port 1010 to seal against shaft 5140 when shaft 5140 is inserted through port 1010.

FIG. 2C illustrates sealing member 1000 having been installed on apparatus 500. Note that the implant 10 does not need to be attached to the apparatus 500 until after sealing member 1000 has been installed on apparatus 500 and therefore the ports 1008, 1010 do not need to be designed to accommodate the passage of the implant 10 therethrough. FIG. 2D illustrates the working end portions 4010 and 5010 having been inserted into introducer cannula 310L and sealing member 1000 having been attached to the proximal end of introducer 310L to seal off the proximal end of the central lumen of cannula 310L, forming a seal between the inner wall of the cannula 310L/handle 590h and the shafts 4140, 5140. Attachment members 1004 have been snap fitted to the handle/end cap 590h of introducer cannula 310L and seal 1006 (not shown in FIG. 2D) forms a seal against the inner wall of the cannula 310L. The first elastic valve 1012 forms a seal with the shaft 4140 and the elastic seal 1013 forms a seal with the shaft 5140.

FIG. 2E illustrates the capability of axially advancing the apparatus 500 relative to introducer cannula 310L to extend the working ends 4010, 5010 distally of the distal end of cannula 310L while maintaining the seal between the proximal end of the cannula 310L and the shafts 5140, 4140. FIG. 2F illustrates the capability of elastic valve 1012 to expand while still conforming to the cross-sectional shape of the portion of the instrument extending therethrough. In FIG. 2F, attachment tool 4000 is being withdrawn form introducer cannula 310L. As the shaft 4140 clears the valve 1012 and working end 4101 enters the valve 4010, the elastic valve expands to conform to the cross-sectional shape and dimensions of the working end 4010, while maintaining a seal therewith. FIG. 2G illustrates use of closure member 1014 to positively seal off the elastic valve 1012 after complete removal of the attachment tool 4000 therefrom. As shown, closure member 1014 comprises a snap clip having a hinge 1014h at one end and a clasp 1014c at the other end to releasably lock the opposing arms 1014a together to compresses the jaws of the “duckbill” together, thereby sealing off the opening of the duckbill elastic valve 1012. Alternative mechanical closure members 1014 may be substituted to accomplish this function.

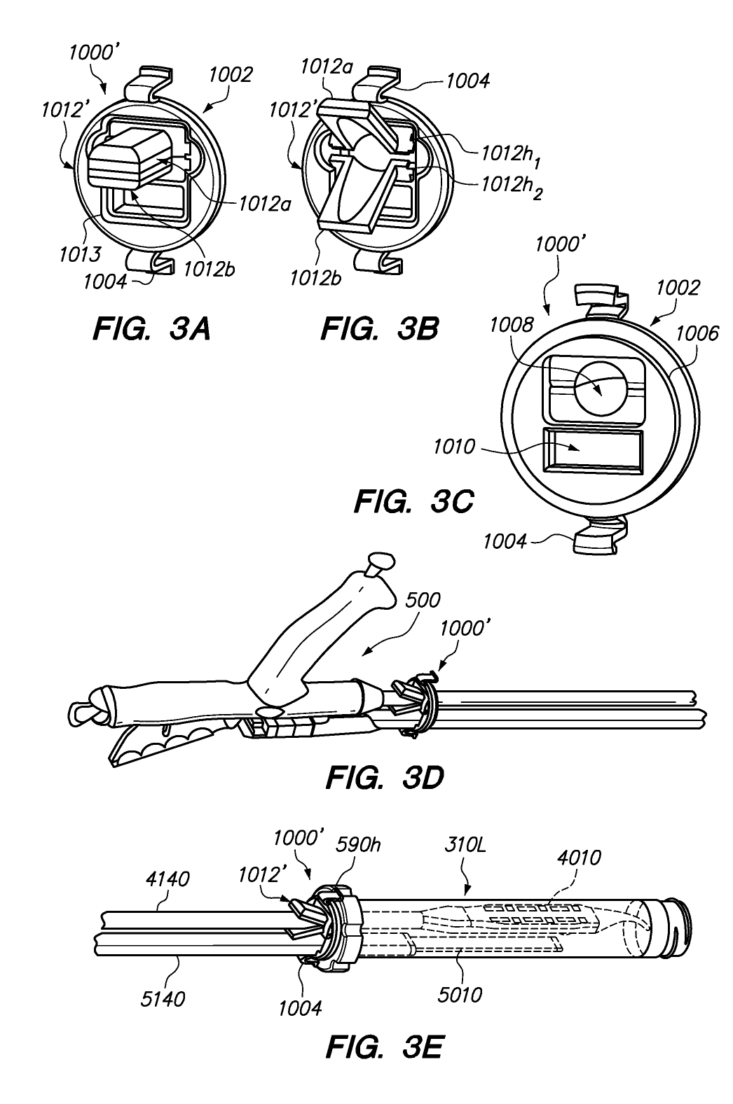

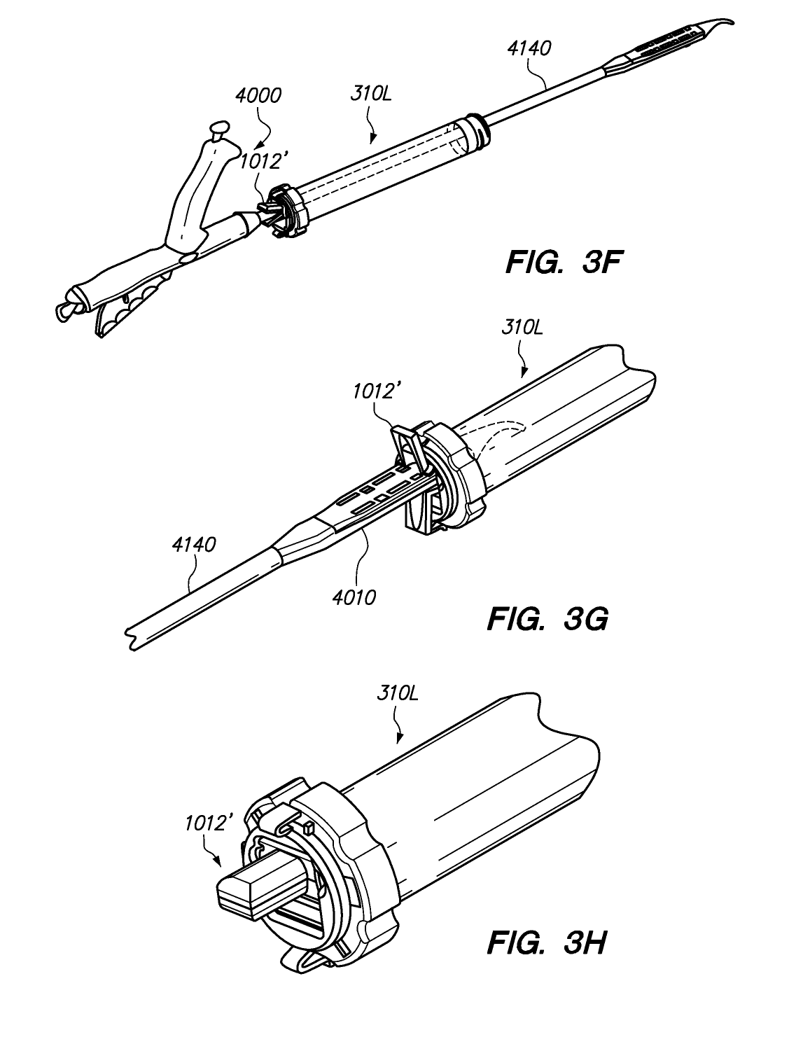

FIGS. 3A-3B show a proximal end perspective view with the first valve in a closed configuration, and with the first valve in an open configuration, respectively, of another embodiment of a sealing member 1000′ according to the present invention. FIG. 3C shows a distal end perspective view of sealing member 1000′. Sealing member 1000′ is configured and dimensioned to form a seal between a tubular member such as introducer cannula 310L and apparatus 500 and/or one or more other surgical tools or instruments. Sealing member 1000′ includes a main body 1002 having a generally circular cross-sectional configuration. Attachment members 1004 are provided to facilitate attachment of the sealing member 1000′ to introducer cannula 310L or other tubular member where a seal is to be formed. As shown, attachment members 1004 are clips that form a snap fit with the distal end of the tubular member or an end cap/handle thereof.

A sealing ring 1006 (such as an O-ring or the like) is provided on the distal end portion of main body 1000′. The distal end portion of the main body 1000′ is configured to slide within the lumen of the introducer cannula 310L with a close fit and sealing ring 1006 forms a friction fit with the inner wall of the introducer cannula 310L, thereby forming a seal between the sealing member main body 1000′ and the inner wall of the introducer cannula.

Main body 1000′ is provided with two ports: a first port 1008 is configured and dimensioned to receive the working end 4010 and shaft 4140 of attachment tool 4000 therethrough; and a second port 1010 is configured and dimensioned to receive the working end 5010 and shall 5140 of suturing tool 5000 therethrough. Because shaft 4140 has a different cross-sectional shape than working end 4010, a first valve 1012′ is sealed around the perimeter of first port 1008 and extends therefrom to provide a sealing valve having variable cross-sectional shapes and dimensions. In the embodiment of FIG. 3A, the first valve 1012′ includes a pair of hinged valve leaflets or doors 1012a, 1012b. In the open configuration shown in FIGS. 3B and 3D, when the doors 1012a, 1012b are open, the ends of the doors 1012a, 1012b closest to the hinges 1012h1, 1012h2 form a circle that seals around the shaft 4140. As shown, first valve 1012′ extends proximally of the proximal end of main body 1000′. Alternatively, first valve 1012′ could extend distally of the proximal end of main body 1000′, but the configuration shown in FIG. 3A is preferred. The leaflets 1012a, 1012b are rotationally biased about the hinges 1012h1, 1021h2 to the closed configuration shown in FIG. 3A. Accordingly, when nothing is inserted through the port 1008 and between the leaflets 1012a, 1012b, the leaflets 1012a, 1012b automatically close, thereby sealing off the port 1008. Although not shown, a clip 1014 or other locking device may be configured to be locked over leaflets 1012a, 1012b and may optionally be used to lock the sealed off configuration shown in FIG. 3A. A seal 1013 is formed around the perimeter of port 1010 to seal against shaft 5140 when shaft 5140 is inserted through port 1010.

FIG. 3D illustrates sealing member 1000′ having been installed on apparatus 500. Note that the implant 10 does not need to be attached to the apparatus 500 until after sealing member 1000′ has been installed on apparatus 500 and therefore the ports 1008, 1010 do not need to be designed to accommodate the passage of the implant 10 therethrough. FIG. 3E illustrates the working end portions 4010 and 5010 having been inserted into introducer cannula 310L and sealing member 1000′ having been attached to the proximal end of introducer 310L to seal off the proximal end of the central lumen of cannula 310L, forming a seal between the inner wall of the cannula 310L/handle 590h and the shafts 4140, 5140. Attachment members 1004 have been snap fitted to the handle/end cap 590h of introducer cannula 310L and seal 1006 (not shown in FIG. 3E) forms a seal against the inner wall of the cannula 310L. The first valve 1012′ forms a seal with the shaft 4140 and the seal 1013 forms a seal with the shaft 5140.

FIG. 3F illustrates the attachment tool 4000, with working end 4010 having been axially advanced through introducer cannula 310L to extend the working end 4010 distally of the distal end of cannula 310L while maintaining the seal between the proximal end of the cannula 310L and the shafts 4140 via valve 1012′. In the first working configuration, valve 1012′ has a substantially round opening that forms a seal around the shaft 4140 of tool 4000. In the second working configuration, illustrated in FIG. 3G, which is assumed whenever the working end 4010 is inserted through or withdrawn from opening 1008, valve 1012′ forms a substantially rectangular shape that forms a seal with the working end 4010. Upon withdrawal of the tool 4000 so that nothing is extending through the opening 1008 or valve 1012′, valve 1012′ assumes a closed configuration, as shown in FIG. 3H. The closed configuration seals off the opening 1008, thereby maintaining the opening closed off to substantially prevent insufflation gas from escaping therethrough, for example.

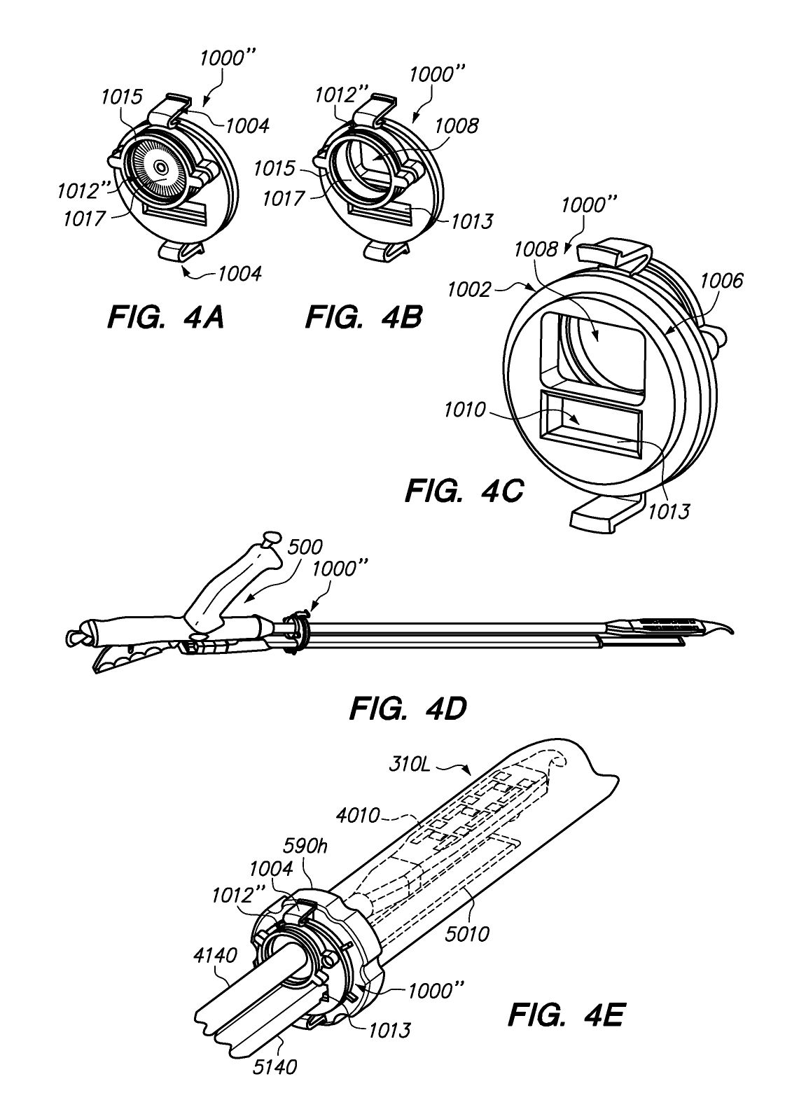

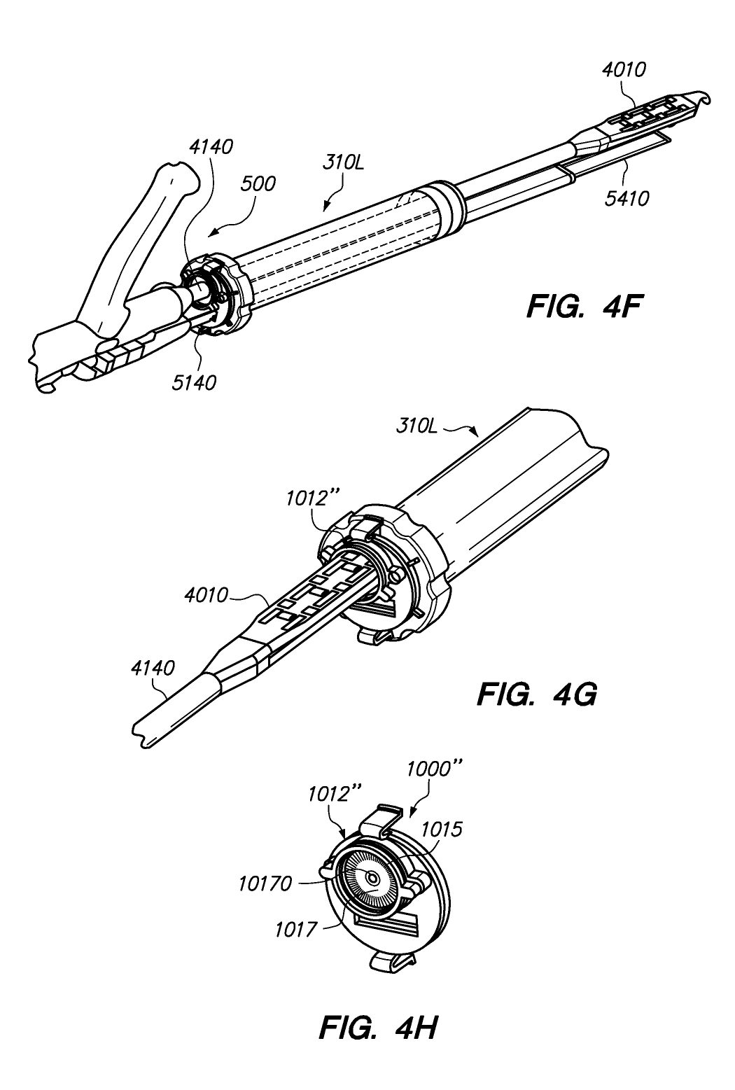

FIGS. 4A-4B show a proximal end perspective view of another embodiment of a sealing member 1000″, with the first valve 1012″ in a closed configuration, and with the first valve 1012″ in an open configuration, respectively. The seal 1013 seals around the shaft 5140 and is a simple flap valve that automatically closes when the shaft 5140 is removed from the opening, thereby sealing off the opening. FIG. 4C shows a distal end perspective view of sealing member 1000″. Sealing member 1000″ is configured and dimensioned to form a seal between a tubular member such as introducer cannula 310L and apparatus 500 and/or one or more other surgical tools or instruments. Sealing member 1000″ includes a main body 1002 having a generally circular cross-sectional configuration. Attachment members 1004 are provided to facilitate attachment of the sealing member 1000″ to introducer cannula 310L or other tubular member where a seal is to be formed. As shown, attachment members 1004 are clips that form a snap fit with the distal end of the tubular member or an end cap/handle thereof.

A sealing ring 1006 (such as an O-ring or the like) is provided on the distal end portion of main body 1000″. The distal end portion of the main body 1000″ is configured to slide within the lumen of the introducer cannula 310L with a close fit and sealing ring 1006 forms a friction fit with the inner wall of the introducer cannula 310L, thereby forming a seal between the sealing member main body 1000″ and the inner wall of the introducer cannula.

Main body 1000″ is provided with two ports: a first port 1008 is configured and dimensioned to receive the working end 4010 and shaft 4140 of attachment tool 4000 therethrough; and a second port 1010 is configured and dimensioned to receive the working end 5010 and shaft 5140 of suturing tool 5000 therethrough. Because shaft 4140 has a different cross-sectional shape than working end 4010, a first valve 1012″ is provided to assume variably-sized openings. Valve 1012″ includes a rotating hub 1015 that is rotatable clockwise as well as counterclockwise relative to main body 1000″. By rotating in a first direction, a flexible sleeve 1017 is twisted down toward a smaller opening configuration, such as illustrated in FIG. 4A, for example. The flexible sleeve 1017 extends between two cylindrical hubs. When the hubs are relatively rotated in opposite directions then the sleeve twists in a closing direction. By reversing the directions of relative rotations, the sleeve 1017 is opened, as in FIG. 4B. It is noted that sleeve 1017 is variably and continuously adjustable. For example, in FIG. 4A, sleeve 1017 is closed down to an extent where it would seal against the shaft of a small endoscope such as a 5 mm endoscope. However, sleeve 1017 can be closed down to other sizes, such as one where it seals against shaft 4140 (e.g., see FIG. 4F) or against working end 4010. FIG. 4B shows the rotating hub 1015 having been rotated in the opposite direction as far as possible to open the sleeve 1017 fully. Thus, FIG. 4B shows valve 1012″ in the fully open position. Sleeve 1017 is continuously adjustable to vary the opening from the fully open position to any smaller size opening, and can even be rotated to completely seal off the opening. A seal 1013 is formed around the perimeter of port 1010 to seal against shaft 5140 when shaft 5140 is inserted through port 1010. FIGS. 4A-4B show seal 1013 in a fully closed configuration and FIG. 4C shows valve 1013 in a fully open configuration. Seal 1013 is variably adjustable and may be partially open to form an opening smaller than that shown in FIG. 4C.

FIG. 4D illustrates sealing member 1000″ having been installed on apparatus 500. Note that the implant 10 does not need to be attached to the apparatus 500 until after sealing member 1000″ has been installed on apparatus 500 and therefore the ports 1008, 1010 do not need to be designed to accommodate the passage of the implant 10 therethrough. FIG. 4E illustrates the working end portions 4010 and 5010 having been inserted into introducer cannula 310L and sealing member 1000″ having been attached to the proximal end of introducer 310L to seal off the proximal end of the central lumen of cannula 310L, forming a seal between the inner wall of the cannula 310L/handle 590h and the shafts 4140, 5140. Attachment members 1004 have been snap fitted to the handle/end cap 590h of introducer cannula 310L and seal 1006 (not shown in FIG. 4E) forms a seal against the inner wall of the cannula 310L. The first valve 1012″ forms a seal with the shaft 4140 and the seal 1013 forms a seal with the shaft 5140.

FIG. 4F illustrates the capability of axially advancing the apparatus 500 relative to introducer cannula 310L to extend the working ends 4010, 5010 distally of the distal end of cannula 310L while maintaining the seal between the proximal end of the cannula 310L and the shafts 5140, 4140.

FIG. 4G illustrates the withdrawal of working end 4010 from introducer cannula 310L and through valve 1012″. As working end 4010 is pulled out of the rotational valve 1012″, the sleeve 1017 untwists to a more open configuration, allowing the working end 4010 to be removed. Sleeve 1017 may untwist automatically after removal of the working end 4010. Alternatively a latch (not shown) may be provided that the operator releases to unlock the current position of the sleeve 1017 and allow it to unwind. FIG. 4H illustrates a configuration where rotating hub has been rotated to close the rotational valve 1012″ so that the sleeve 1017 twists down to form a very small opening 1017o. In this configuration, an endoscope (such as a 5 mm endoscope or larger) can be inserted through opening 1017o so that sleeve 1017 forms a seal against the shaft of the endoscope. Sleeve 1017 may be made of silicone, for example. The rotational valve can be closed by further rotating hum 1015, when nothing is inserted through the opening 1017o, to completely close the sleeve to prevent gas/fluids from escaping from the introducer cannula 310L. This allows insufflation pressure to be maintained in the abdominal cavity even when the introducer remains inserted therein and no tools are extending through the sealing member 1000″.

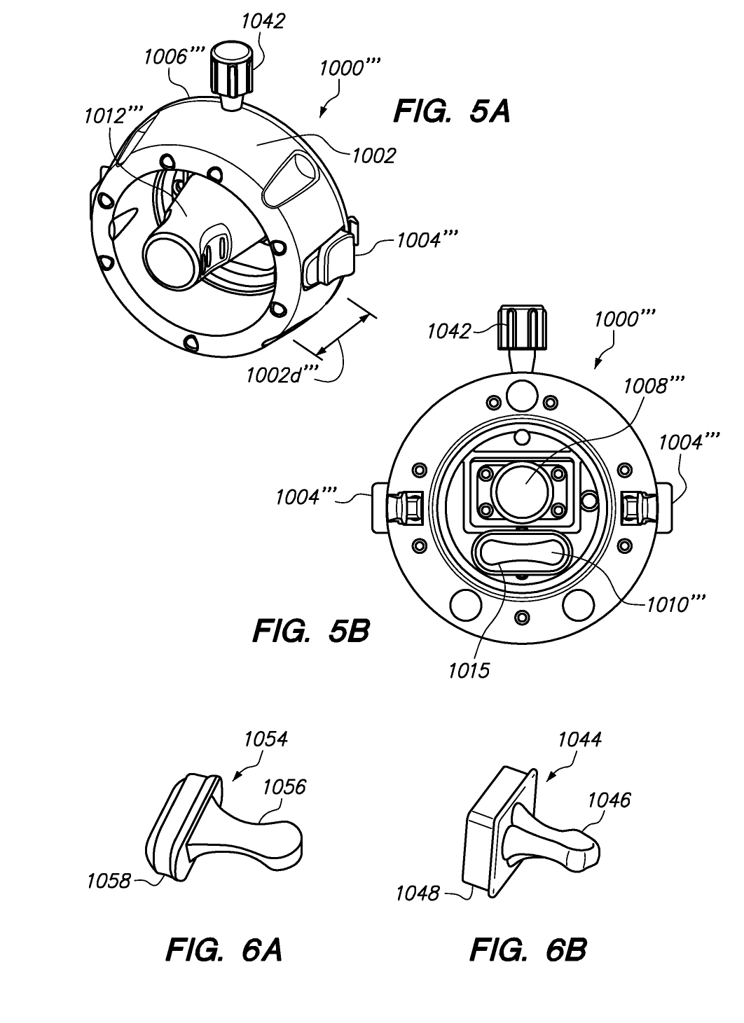

FIGS. 5A-5B show a proximal end perspective view and a distal end view of a sealing member 1000′″, respectively, according to another embodiment of the present invention. Sealing member 1000′″ is configured and dimensioned to form a seal between a tubular member such as introducer cannula 310L and apparatus 500 and/or one or more other surgical tools or instruments. Sealing member 1000′″ includes a main body 1002′″ having a generally circular cross-sectional configuration and a greater depth (thickness) dimension 1002d′″ than previously described embodiments, that facilitates easier grasping and manipulation by the surgeon/user. Attachment members 1004′″ are provided to facilitate attachment of the sealing member 1000′″ to introducer cannula 310L or other tubular member where a seal is to be formed. As shown, attachment members 1004′″ are clips that form a snap fit with the distal end of the tubular member or an end cap thereof.

A sealing ring 1006′″ (such as an extension made of an elastomeric material, or a more rigid material with an O-ring or the like) is provided on the distal end portion of main body 1000′″. The distal end portion of the main body 1000′″ is configured to slide within the lumen of the introducer cannula 310L with a close fit and sealing ring 1006′″ forms a friction fit with the inner wall of the introducer cannula 310L, thereby forming a seal between the sealing member body 1000′″ and the inner wall of the introducer cannula 310L.

Main body 1000′″ is provided with two ports: a first port 1008′″ configured and dimensioned to receive the working end 4010 and shaft 4140 of attachment tool 4000 therethrough; a second port 1010′″ configured and dimensioned to receive the working end 5010 and shaft 5140 of suturing tool 5000 therethrough. Optionally, a third port 1042 may be provided with this embodiment (or with any of embodiments 1000, 1000′ or 1000″ in like manner) to enable insufflation gas to be inputted therethrough, from a location proximal of sealing member 1000′″ to a location distal of sealing member 1000′″. Optionally a fourth port may be present to allow the implant tubing to pass through the seal without allowing leakage. Seal 1012′″ is configured to create a sliding seal around the shaft 4140, and a seal to main body 1000′″. When the attachment tool 4000 is removed, seal 1012′″ is configured to slide along the shaft 4140 and, when the end effector 4010 collides with the seal 1012′″, the seal 1012′″ is configured to release from the main body 1000′″, allowing the attachment tool 4000 to be completely removed from the body of the patient. This detachment/release of the seal 1021′″ from the main body 1000′″ leaves a hole in the main body 1000′″ which is plugged with plug 1044 to regain a seal and insufflation. As shown, seal 1012′″ has a conical shape. Elastic seal 1010′″ is sealed around the perimeter of first port 1008′″ and extends distally therefrom. As shown, elastic seal 1012′″ extends distally of the port 1008′″. Alternatively, seal 1012′″ could extend proximally of port 1008′″.

Although not shown, sealing member 1000′″ is installed on apparatus 500 in similar manner to that shown in FIG. 2C, prior to inserting apparatus 500 into cannula 310L. Note that the implant 10 does not need to be attached to the apparatus 500 until after sealing member 1000′″ has been installed on apparatus 500 and therefore the ports 1008′″, 1010′″ do not need to be designed to accommodate the passage of the implant 10 therethrough.

In this embodiment, when attachment tool 4000 is withdrawn from introducer cannula 310L, as shaft 4140 clears the elastic member 1012′″ working end 4101 contacts the tapered-down distal end of seal 1012′″, As the working end is withdrawn from the port 1008, it will typically pull the seal 1012′″ along with it, at which time the seal 1012′″ detaches from the port 1008′″. In order to seal off the port 1008′″ again, plug 1044 (FIG. 6B) is inserted into the port 1008′″, where it functions as a stopper by closing and sealing off the opening 1008′″. Plug 1044 includes a handle 1046 that facilitates grasping by a user, and a main body 1048 dimensioned to fit in port 1008′″ and form a seal therewith. Plug 1044 may be made of a substantially rigid plastic or rubber, and port 1008′″ may include an elastic seal that deforms elastically around body 1048 as body 1048 is inserted into the port, to form an airtight, pressure-tight seal.

When suturing/stitching tool 5000 is withdrawn from introducer cannula 310L, in order to seal off the port 1010′″ again, plug 1054 (FIG. 6A) is inserted into the port 1010′″, where it functions as a stopper by closing and sealing off the opening 1010′″. Plug 1054 includes a handle 1056 that facilitates grasping by a user, and a main body 1058 dimensioned to fit in port 1010′″ and form a seal therewith. Plug 1054 may be made of a substantially rigid plastic or rubber, and port 1010′″ (as well as any of ports 1010, 1010′ and 1010″ described above) may include an elastic seal 1015 that deforms elastically around body 1058 as body 1058 is inserted into the port, to form an airtight, pressure-tight seal. Optionally, these features can allow the attachment tool 4000 and suturing/stitching tool 5000 to be reintroduce into the abdomen; or can allow another device or tool that is configured to establish a seal within 1008′″ or 1010′″ to be introduced, for example, an endoscope or graspers, or two devices/tools at one time can be introduced/reintroduced.**

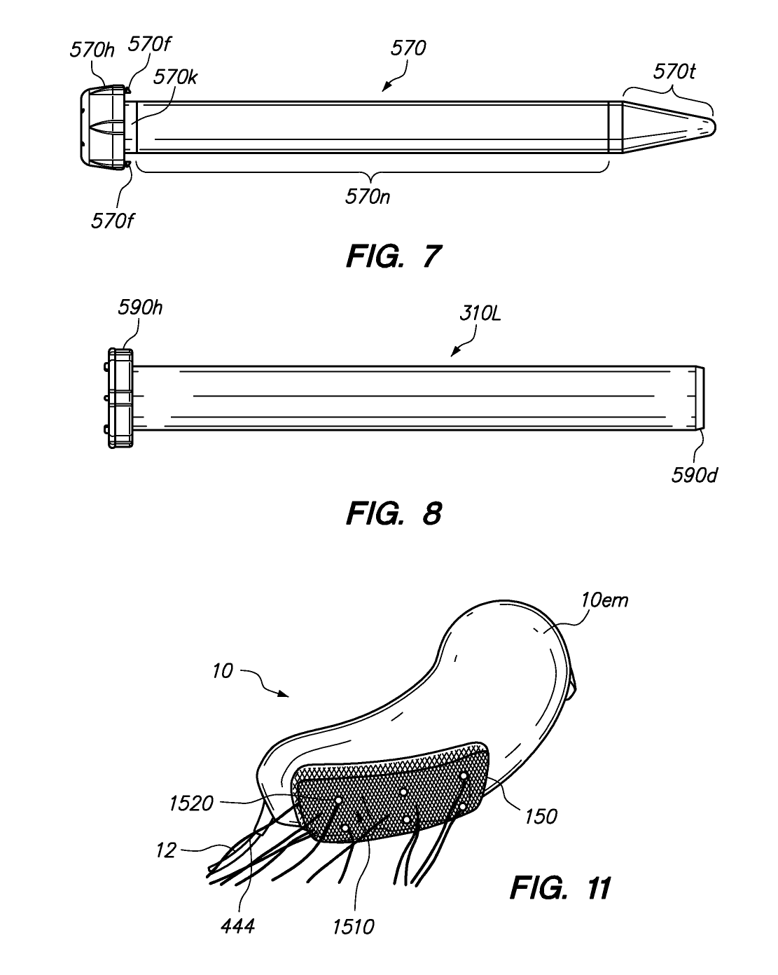

FIG. 7 shows one embodiment of a dilator 570 that may be used in procedures according to the present invention as described herein and FIG. 8 shows one embodiment of an introducer cannula (large cannula) 310L that can be used in procedures according to the present invention as described herein. These tools can be used, inter alia, to enlarge an opening formed through the fascia leading into the abdominal cavity. However, these techniques are not limited to enlarging an opening into the abdominal cavity, as they can also be used to enlarge an opening into the thoracic cavity, or to enlarge another opening leading into the patient.

Dilator 570 is tapered, and is similar to the dilators 570 described in application Ser. Nos. 12/474,226; 12/473,818; and 12/474,118, but lacks threads on the tapered portion 570t and is instead smooth surfaced along the tapered portion. Additionally, the outside diameter of the non-tapered portion 570n is somewhat smaller than previous embodiments and the overall length of the dilator shown in FIG. 7 is somewhat greater than that of previous embodiments. Still further, that is no opening at the distal end of the tapered portion, so that there is not a central lumen that extends all the way through the tool, from proximal end to distal end. By closing off the distal end, this equips the dilator to prevent loss (or at least rapid or substantial loss) of insufflation pressure in the abdominal cavity when the dilator 570 and introducer cannula 310L extend into the abdominal cavity during procedures performed under insufflation as described in detail below.

In at least one embodiment, the tapered portion has an angle of taper such that the outer surface of the tapered portion 570t relative to a central longitudinal axis of the dilator 570 is in the range of about seven degrees to about 13 degrees, typically about eight degrees to about 12 degrees. In one embodiment, the angle was about 10.5 degrees (or 21 degrees measured from outer surface to opposite outer surface of the cone).

In at least one embodiment, the outside diameter of the non-tapered portion is about 1.35″ to about 1.75″. The distal end of dilator 570, where the tapered portion begins has an outside diameter of about 0.6″ to about 0.7″ and tapers to the cross-sectional dimension of the non-tapered section 570n, which may, for example, have an outside diameter of about 1.0 inches to about 1.5 inches. In another example, the outside diameter of the non-tapered portion 570n was about 1.2 inches. Dilator 570 and introducer cannula 310L each can be made from one or more of the following materials: a relatively rigid, but optionally lubricious polymer, such as DELRIN® (acetal copolymer) or other acetal copolymer, or other suitable biocompatible polymer, such as an injection moldable polycarbonate, glass-filled polycarbonate, glass-filled nylon, Grilamid® (semi-lubricious nylon product) Grivory® (semi-lubricious nylon product), polyetheretherketone (PEEK), Teflon® (polytetrafluoroethylene) or other injection molded, biocompatible plastic. Either or both dilator 570 and introducer cannula 310L may be provided with or without a radiopaque filler or radiopaque marker band.

Dilator 570 additionally includes an enlarged handle 570h at a proximal end thereof that is configured to be grasped by a user to facilitate an increase in the amount of torque and/or axial force the user can apply to the dilator 570 by rotating and/or pushing on handle 570h. Thus, handle 570h has a larger outside diameter than the non-tapered cylindrical portion 570n of dilator 570. Further, handle 570h can be provided with knurls 570k or other features that render handle 570h less smooth or otherwise increase friction, to prevent the user's hand from slipping during torquing.

The introducer cannula 310L of FIG. 8 is configured to slide over dilator 570 with a close, but freely sliding fit (e.g., inside diameter of introducer/large cannula 310L is about 0.005″±about 0.002″ greater than outside diameter of portion 570n and introducer cannula 310L has a length such that when handle 590h contacts handle 570h, the tapered portion 570t of dilator 570 extends distally of the distal end of introducer cannula 310L in the same manner as described and shown in the previous applications incorporated by reference above. In another embodiment, the close, but freely sliding fit is provided wherein the inside diameter of large cannula 310L is about 0.012″±about 0.005″ greater than outside diameter of portion 570n. In one embodiment, where the dilator had a length of about 8.67″, the portion 570n had an outside diameter of about 0.995″, the large cannula 310L had a length of about 6.375″, an inside diameter of about 1.055″ and an outside diameter of about 1.105″). In another embodiment, where the dilator had a length of about 16.16″, and inside diameter of about 0.505″ and the portion 570n had an outside diameter of about 1.588″, the large cannula 310L had a length of about 11.855″, an inside diameter of about 1.610″ and an outside diameter of about 1.690″. In another particular embodiment, the dilator had a length of about 8.67″ and the same inside diameter as the previous embodiments, but an outside diameter of about 1.060″ and the large cannula had a length of about 6.375″, an inside diameter of about 1.065″ and an outside diameter of about 1.115″). In another embodiment the outside diameter of cannula 310L is about 3.4 cm. In all embodiments, the inside diameter of the distal end of the large cannula 310L forms a close fit with the outside diameter of the portion of the dilator 570 that it interfaces with to allow free sliding between the components, but to prevent snagging of tissue between the distal end of large cannula 310L and dilator 570 as these components are inserted into the body. Proximal of this interface, the dilator tubing can be much smaller and could even be a solid rod having an outer diameter much less than the inner diameter of the large cannula 310 (in one example, about 0.5″ outer diameter), thus leaving a large gap between the inner walls of the large cannula 310L and the outer diameter of the dilator tubing, at locations proximal of the distal end interface described above. The distal end portion of introducer cannula 310L may comprise a radiopaque material or may be provided with a radiopaque feature for enhanced visibility under fluoroscopy. Likewise, the distal end portion of dilator 570 may comprise a radiopaque material or may be provided with a radiopaque feature for enhanced visibility under fluoroscopy.

Large cannula 310L, like dilator 570, has a smooth outer surface to render it less traumatic to tissues as it is inserted into the body. Handle 570h may be provided with at least one fastening component 570f and handle 590h may be provided with at least one mating fastening component (not shown, in FIG. 8, but shown in previous applications incorporated herein), one for each respective fastening component 570f. As shown in FIG. 7, handle 570h includes two male fastening components 570f. However, one or more than two such components may be provided on handle 570h, with corresponding, mating components in handle 590h. Further, the male component(s) can be provided on handle 590h and the female components can be provided in handle 570h. Still further, although bayonet couplings 570f and mating female receptacles 590f are used in the embodiments shown in FIGS. 5-6, alternative mating components may be used, such as shafts with ball and detent arrangements, or any of a number of mating, releasable mechanical fixtures. The mating mechanical members, when connected, maintain the large cannula 310L fixed relative to the dilator 570, both in the axial direction, as well as rotationally. A release mechanism may be provided that the user can actuate to release the mechanical fixation members and then the operator can remove the dilator 570 from the large cannula 310L in a manner shown and described in application Ser. No. 12/474,226. Handles 570h, 590h can have substantially the same size/outside diameter, but this is not necessary.

The distal end portion of introducer cannula 310L may be chamfered 590d so that it tapers towards the dilator 570 when assembled thereover, thereby further reducing the risk of snagging tissue (e.g., fascia) as the tools are advanced into the body. Alternatively, the tip 590d may be flexible and tapered to a smaller diameter to create intimate contact and smooth transition with the dilator 570. In this embodiment, the tip 590d could be composed of an elastomeric material or a more rigid material where the tip 590d is radially interrupted to allow the stiffer material to flex radially outwards to allow an interference fit that slides under low force.

Large cannula/introducer 310L includes a transparent main body tube with a handle portion 590h. The handle 590h and distal end portion of introducer 310L may be opaque, but alternatively, can be transparent. Preferably, the inside wall of the main body tube is coated with a lubricious coating, such as LUBRILAST™, from AST Products.

In the dilator embodiment of FIG. 7, non-tapered portion 570n is transparent. Tapered portion 572 is opaque and handle 570h is opaque. The transparent tubes 310t and 570n can be extruded parts (e.g., extruded from polycarbonate) and the opaque components 590h, 590t, 570t and 570h can be molded (e.g., molded from polycarbonate).

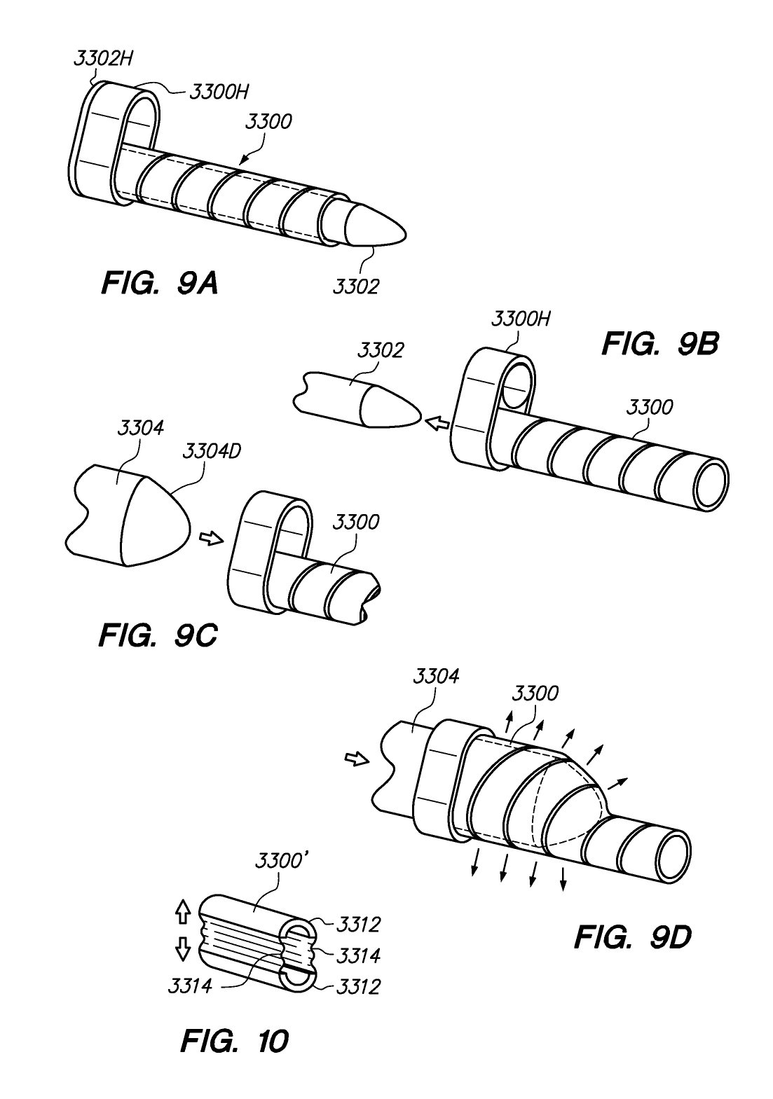

FIGS. 9A-9D illustrate an introducer/cannula 3300 that is insertable into a patient in a first configuration and then is expandable to a second expanded configuration. In a first or initial configuration (FIGS. 9A-9C), introducer/cannula 3300 has a cross-sectional area that is significantly smaller than when introducer/cannula 3300 is in an expanded, configuration (FIG. 9D). FIG. 9A shows introducer/cannula 3300 in the first configuration. Introducer trocar 3302 is shown installed in introducer/cannula 3300 in FIG. 9A, in a configuration ready to be inserted into the abdominal cavity of the patient. As shown in FIG. 9A, trocar 3302 has a circular cross-section and an outer diameter that is only slightly less than the inside diameter of introducer/cannula 3300, so that the trocar 3302 can be readily slid into the lumen of introducer/cannula 3300, but so that the space between the lumen of the introducer/cannula 3300 and the distal portion of trocar 3302 where it extends from the distal end of introducer/cannula 3300 is small, to prevent capturing tissue between the introducer/cannula 3300 and trocar 3302 as they are advanced along the tract into the abdominal cavity. Dimensions of 3300 I.D. and 3302 O.D. may be similar to those discussed with regard to components 570 and 310L of FIGS. 7-8

Optionally, handle 3302H may releasably lock or latch to handle 3300H to help keep the components together as they are being advanced into the patient. Such latching or locking may be performed in the same or equivalent manner to that described with latching or locking 570h and 570f as described herein or in applications incorporated by reference herein.

Once introducer/cannula 3300 has been inserted into its desired position (which may include handle 330H in abutment with the skin of the patient, or in abutment with the fascia or external abdominal wall of the patient, or a position in which handle 330H is proximal of and out of contact with the skin trocar 3302 is withdrawn proximally out of introducer/cannula 3300 (after first releasing the latching or locking between handles 3302H and 3300H, if applicable) as illustrated in FIG. 9B, while introducer/cannula 3300 is held stationary.

Next, enlarging trocar 3304 is inserted into introducer/cannula 3300, as illustrated in FIGS. 9C-9D. As shown in FIG. 9C, enlarging trocar 3302 has an oval cross-section and a cross-sectional area that is substantially greater than the cross-sectional area of introducer/cannula 3300 in the first configuration. The outer perimeter of trocar 3304 is configured and dimensioned to slide within handle 3300H. Dimensions of 3300H I.D. and 3304 O.D. may be similar to those discussed with regard to components 570 and 310L of FIGS. 5-6. The distal tip of trocar 3304 is blunt and tapered so as to be configured to be inserted into the lumen of introducer/cannula 3300. As trocar 3304 is advanced into the lumen of introducer/cannula 3300, it expands introducer/cannula 3300 to the expanded configuration as illustrated in FIG. 9D. Accordingly, the lumen of introducer/cannula 3300 in the expanded configuration is greatly increased in cross-sectional area compared to its cross-sectional area prior to expansion thereof. In the example shown, the expanded lumen of introducer/cannula 3300 is substantially oval in shape (although the present invention is not limited to this shape) and is large enough to receive an endoscope 330 side-by-side of a tool. The introducer/cannula 3300 may be made from a variety of polyurethanes or the like. Once the introducer/cannula 3300 has been expanded as desired (typically over the full length of the tubular shaft), trocar 3304 is removed proximally from the expanded introducer/cannula 3300 and the introducer/cannula 3300 is held stationary during the removal. The introducer/cannula 3300 is then ready to receive an endoscope and tool as described.

FIG. 10 is a partial illustration of a shaft of introducer/cannula 3300′ according to another embodiment of the present invention. In this embodiment, introducer/cannula 3300′ functions in substantially the same manner as introducer/cannula 3300 described above. However, rather than being constructed as described above with regards to FIGS. 9A-9D, the shaft of introducer/cannula 3300′ is constructed with hard shell cannula portions 3312 (which can be made from polycarbonate, for example) and expandable intermediate portions 3314 (which can be made from polyurethane, for example). When in the initial configuration, hard shell components 3313 may abut or nearly abut one another, thereby forming a substantially circular cross-section like the cross-section of 3300 in the initial, unexpanded configuration. After insertion of trocar 3304, the lumen of introducer/cannula 3300′ assumes the expanded shape and configuration as illustrated in FIG. 10.

FIG. 11 illustrates an embodiment of an implantable device 10 according to the present invention, configured for delivery and paragastric, extragastric implantation. Device 10 includes enlargeable member 10em (shown in an enlarged configuration in FIG. 11), a filling tube 12 in fluid communication with enlargeable member 10em and having sufficient length to extend out of an opening formed in a patient, through which the device 10 is delivered, when device 10 has been anchored to a surgical target such as the internal wall surface of the abdominal wall, peritoneum and/or fascia. Device 10 further includes an attachment tab 150 bonded to enlargeable member 10em, and having suture retainers 1520 embedded in a top mesh layer 1510 of attachment tab 150. Sutures 444 extend through the suture retainers 1520. Further details about implants 10 that may be used in practicing the present invention can be found in application Ser. Nos. 12/474,226; 12/473,818; 12/474,118; 11/716,986; 11/716,985; and 11/407,701.

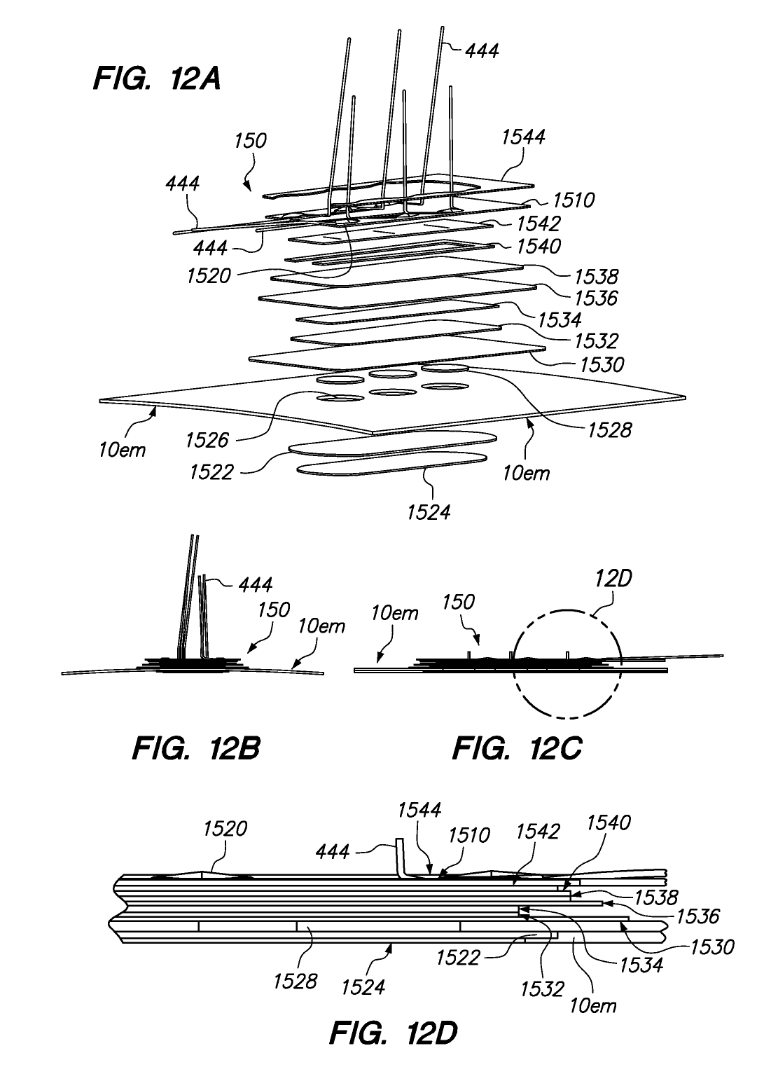

FIG. 12A is an exploded view of another embodiment of attachment tab 150 with an alternative layup arrangement for bonding the attachment tab 150 to the expandable member 10em. In this embodiment, an inner backing layer 1522 comprising a non-vulcanized polymer (preferably, but not limited to non-vulcanized silicone) is provided against the inner surface of the expandable member 10em. Optionally, a reinforced, non-vulcanized inner backing layer 1524 (preferably, but not limited to non-vulcanized silicone reinforced with mesh (reinforced silicone) may be layered against the inner surface of inner backing layer 1522. Openings 1526 are formed through the expandable member 10em. Plugs of non-vulcanized polymer (preferably, but not limited to the same non-vulcanized material that layer 1522 is made of preferably, but not limited to non-vulcanized silicone) are provided to fill the openings 1522 and are placed in the openings so that, when laid up, they contact the outer surface of layer 1522 and the inner surface of main backing and shell layer 1530. Main backing and shell layer 1530 is preferably made of, but not limited to the same non-vulcanized material that plugs 1528 are made of (preferably, but not limited to non-vulcanized silicone). The plugs form an interlock between the bonding members inside and outside when vulcanized. A reinforced backing layer 1532 (made of a material preferably, but not limited to non-vulcanized silicone) is laid on the outside surface of layer 1530.

A wing forming bond layer 1534 (made of a material preferably, but not limited to non-vulcanized silicone) is laid on the outside surface of layer 1532 and a reinforced wing backing layer 1536 (made of a material preferably, but not limited to non-vulcanized silicone reinforced with mesh (i.e., reinforced silicone) is laid on the outside surface of layer 1534. A wing backing layer 1538 (made of a material preferably, but not limited to non-vulcanized silicone) is laid on the outside surface of layer 1538 and a lower ingrowth bond layer 1540 (made of a material preferably, but not limited to non-vulcanized silicone) is laid on the outside surface of layer 1536. As shown, lower ingrowth bond layer 1540 is U-shaped so as to be open at one end to a window for contacting tissue that allows tissue ingrowth into tissue ingrowth encouraging material 1542 (such as velour, or the like) inside the window. Alternatively, layer 1540 does not need to be U-shaped, but could be closed, while still maintaining the tissue ingrowth encouraging window so that layer 1542 can contact the tissue.

A lower ingrowth layer 1542 (preferably, but not necessarily made of a layer of velour, such as DACRON® (polyester fiber) configured and dimensioned to encourage tissue growth into it) is laid on the outside surface of layer 1540 and an upper ingrowth layer 1510 (preferably, but not necessarily formed of ingrowth mesh (e.g., polyethylene terephthalate (PET), having a less dense weave than layer 1540 with less aggressive tissue ingrowth encouragement, resulting in relatively less scarring) configured in a weave pattern to which suture retainers 1520 are fixed and through which sutures 444 are threaded. Sutures 444 weave through one layer of the mesh and are threaded through the mesh to the suture lock and then back out of the mesh. Upper ingrowth layer 1510 is laid over the outer surface of layer 1542. An upper ingrowth bond layer 1544 (preferably, but not necessarily having the same shape as lower ingrowth bond layer 1540 and preferably, but not necessarily formed of non-vulcanized silicone) is laid on upper ingrowth layer 1510 such that it is on the outside of the lower ingrowth layer 1542 and creates contact with wing layers 1538 and 1540. Upper ingrowth bond layer 1540 is U-shaped, or otherwise open at one end to accommodate sliding the suture tool out below it.

With all layers laid up as described, pressure and heat are applied so as to vulcanize the non-vulcanized materials, thereby integrating the previously separate layers into a non-separable vulcanized unit. In one non-limiting embodiment, the layers are vulcanized in a vulcanizing press at about 175° C. and about 100 pounds per square inch (psi) pressure for about twenty minutes, although these temperatures, pressures and times may vary. FIG. 12B illustrates an end view of the attachment tab 150 having been bonded to the expandable member 10em. FIG. 12C is a longitudinal-sectional view, taken along line B-B in FIG. 12B, of the layers having been bonded together to form the attachment tab 150 on the expandable member 10em. FIG. 12D is a detailed view of the vulcanized joint indicated within circle 12D of FIG. 12C. Note that the layers 1524, 1522, 1528, 10em have become mechanically interlocked (through openings 1526) as well as chemically interlocked (through vulcanization). An alternative embodiment could omit the plugs, and instead, during the vulcanization, allow the inner layer to flow into the holes and the outer layer to flow into the holes, thereby connecting and vulcanizing together the inner and outer layers.

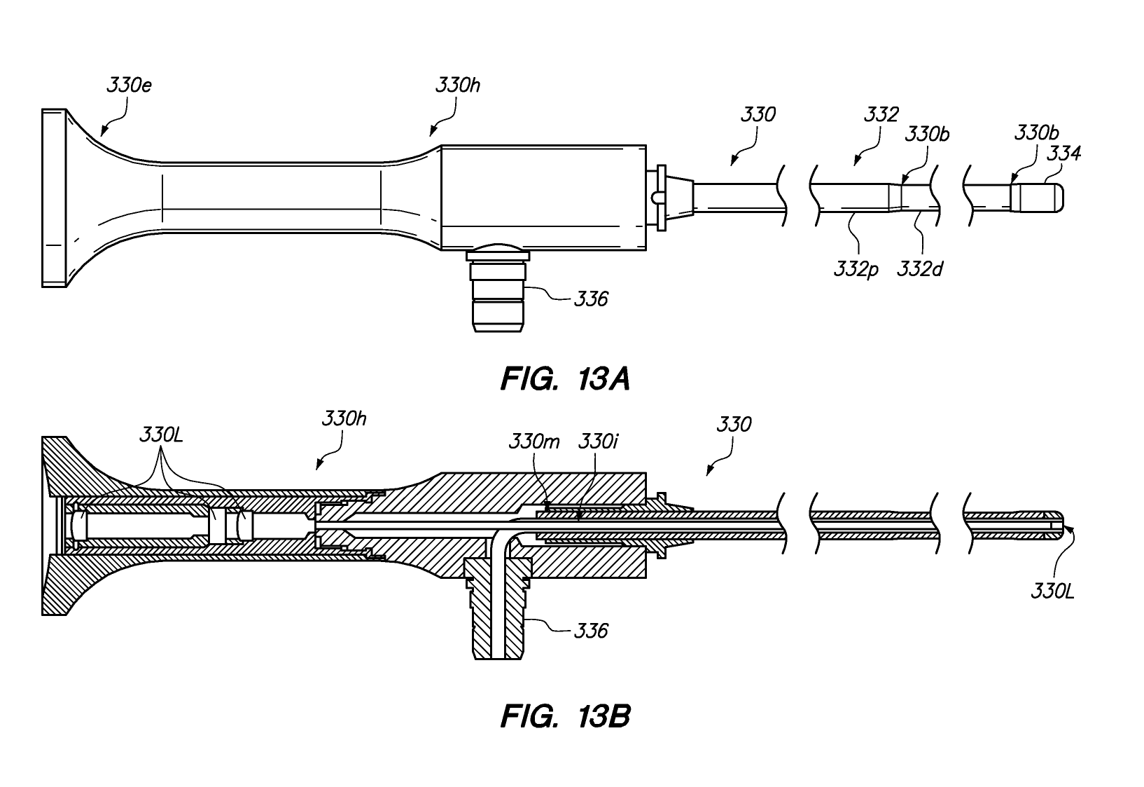

FIG. 13A is a partial view of one embodiment of an endoscope 330 that may be inserted into a port, cannula or tool to provide visualization during performance of one or more steps of a procedure as described herein. Although FIG. 13A shows one embodiment of such an endoscope 330, it is noted that other endoscopes may be substituted therefore to provide visualization during a procedure as described herein. It is further noted that various, different sized endoscopes may be used during different steps or a procedure as described herein. FIG. 13B shows a longitudinal sectional view of the endoscope shown in FIG. 13A. The elongated shaft 332 is only partially shown in FIGS. 13A and 13B, so as to be able to show the views in a larger scale while still allowing them to fit on the page. The proximal portion 332p of shaft 332 is rigid, while the proximal portion 332d is flexible. The lengths of each portion 332p and 332d may vary. In one embodiment, the length of rigid portion was about sixteen inches and the length of the distal portion 332d plus tip 334 was about twenty-seven inches. Alternatively, the elongated shaft 332 may be a rigid shaft over both proximal and distal portions.

Light post 336 is configured in the proximal handle portion 330h of the endoscope. An eve cup 330e is provided at the proximal end of the endoscope 330. Bevels 330b may be provided at the junctures of proximal with distal portions 332p, 332d and distal portion with distal tip 330d, 334. The maximum diameter of the elongated shaft 332 (including tip 334) in one embodiment, is less than or equal to about five millimeters. In the same embodiment, the working length of the elongated shaft 332 (including tip 334) is about 42 inches to about 44 inches. The flexibility of distal flexible portion allows it to bend and therefore the distal tip 334 can be delivered along a non-straight pathway, and it provides imaging to the surgeon so that the surgeon can see where the distal tip 334 is being driven to, and can see the pathway that it is taking, as it travels along the pathway. Additionally, the rigid portion 332p provides some stiffening support to facilitate pushing the distal tip 334 into the patient.

Illumination fibers 330m extend through the main lumen of endoscope 330 and are connectable at a proximal end thereof to a light source (not shown) via light post 336 to deliver light out the distal tip 334 of endoscope 330. Lenses 330L are provided in the main lumen at the location of the distal tip 334 and proximal portion of the handle 330h to provide an image of the light reflected off of the environment as the illumination light exits the tip 334, reflects off objects and is reflected back into tip 334. Imaging fiber(s) connect the distal lens 330L with the proximal lens 330L arrangement in the handle 330h. A camera (not shown) may be connected to the endoscope for providing the ability to display images on a computer screen, provide image prints, etc.

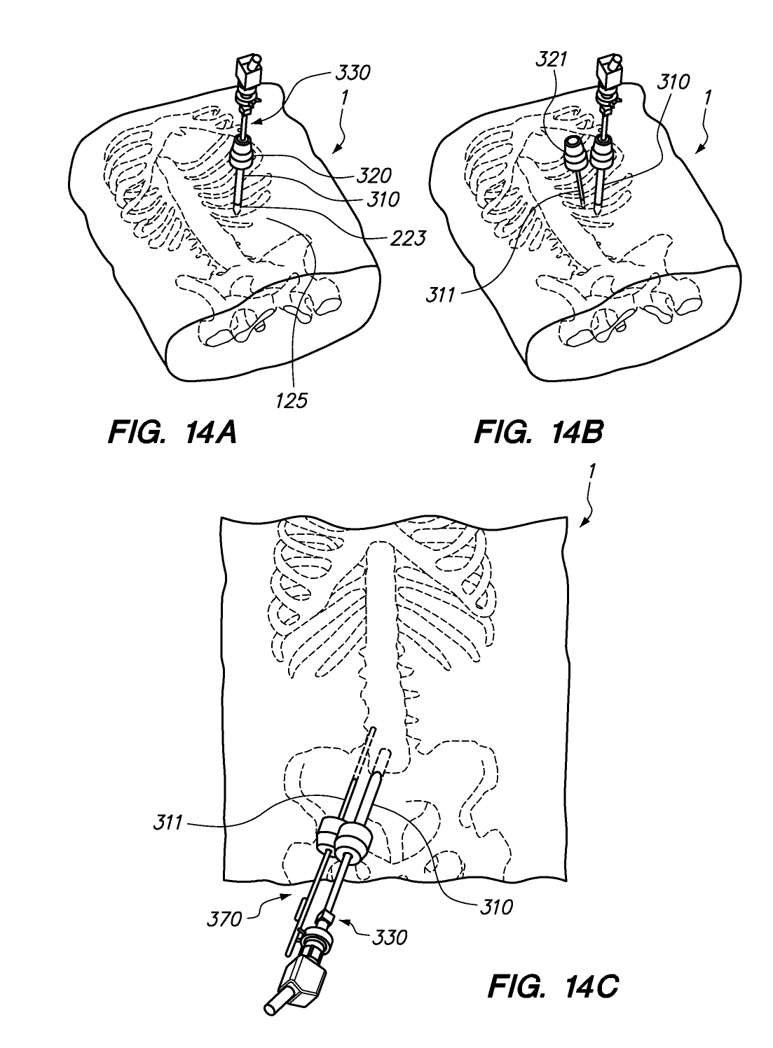

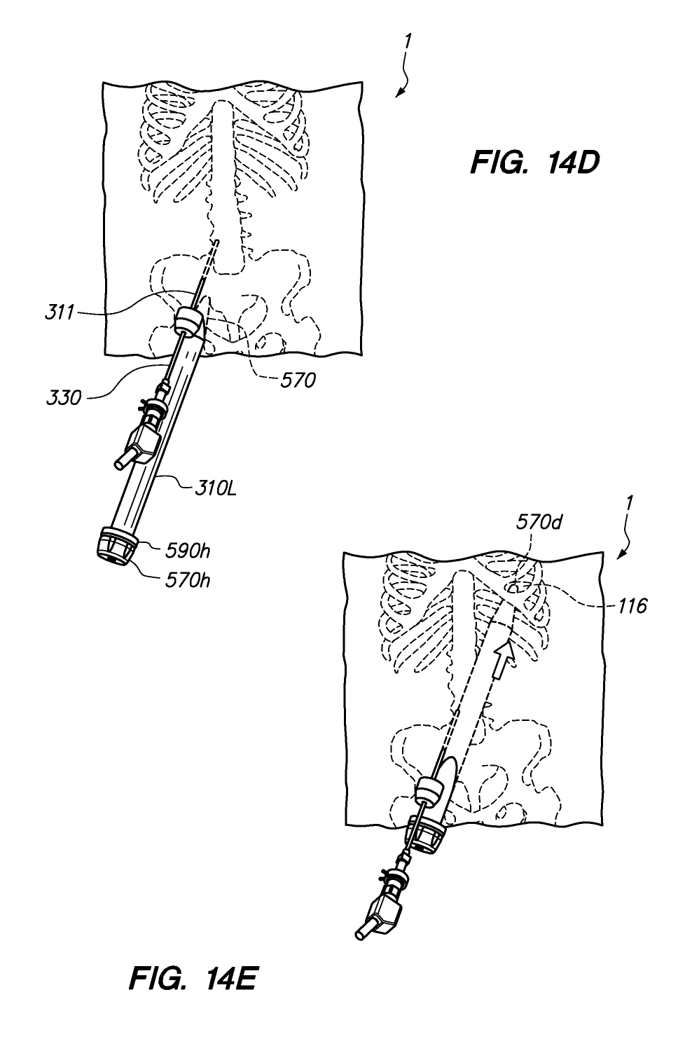

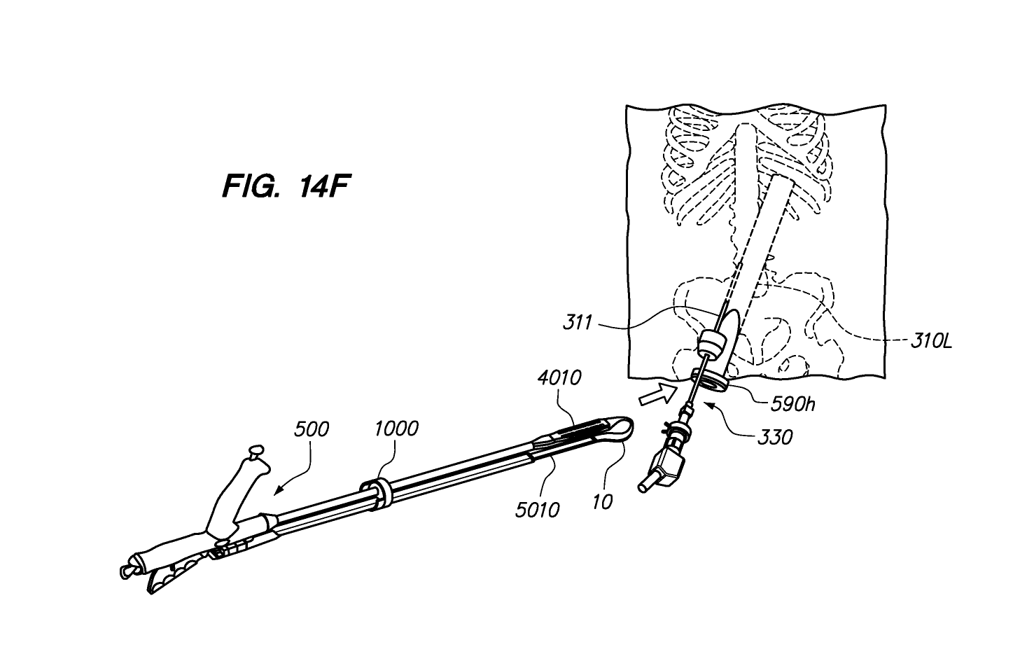

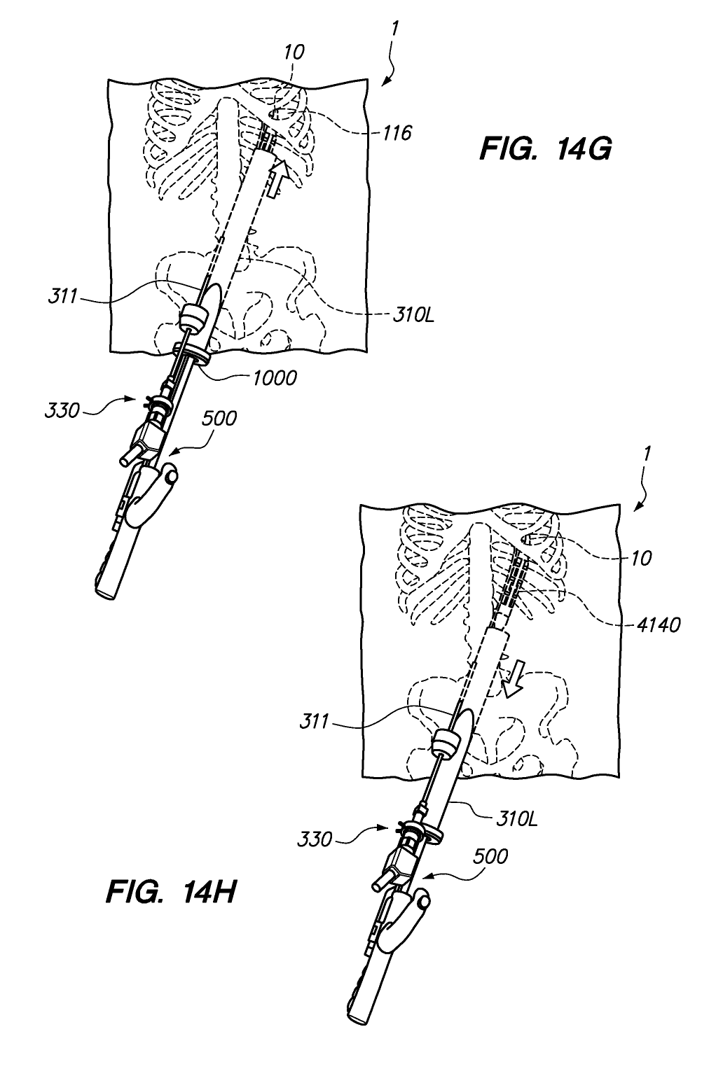

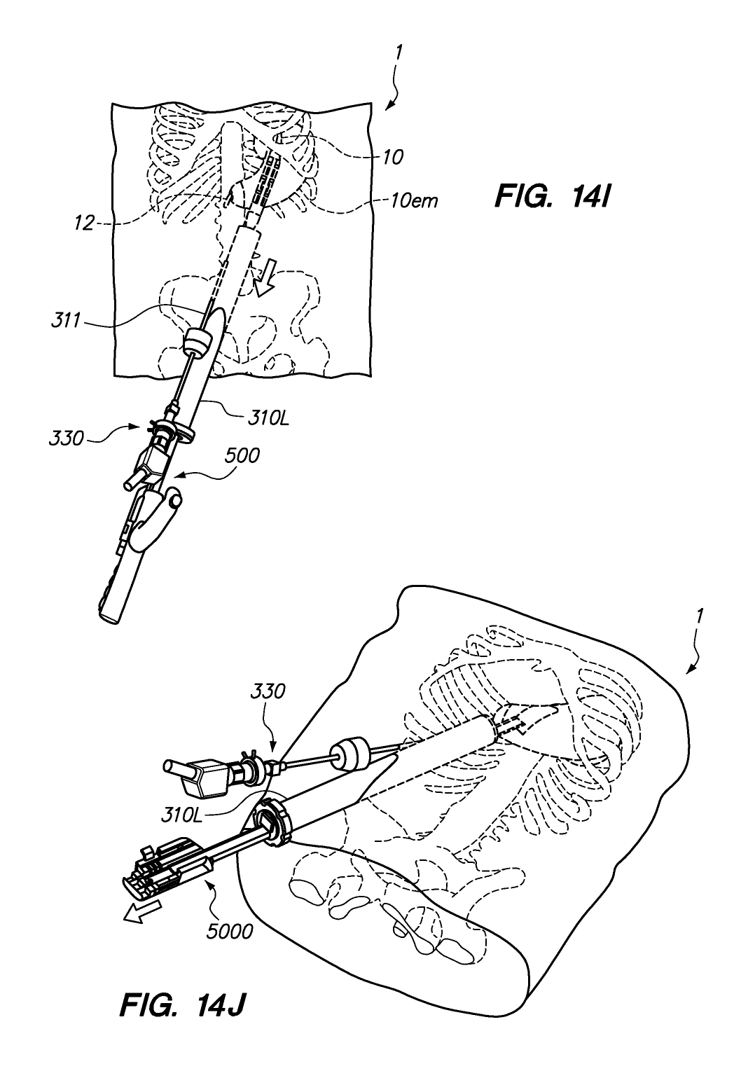

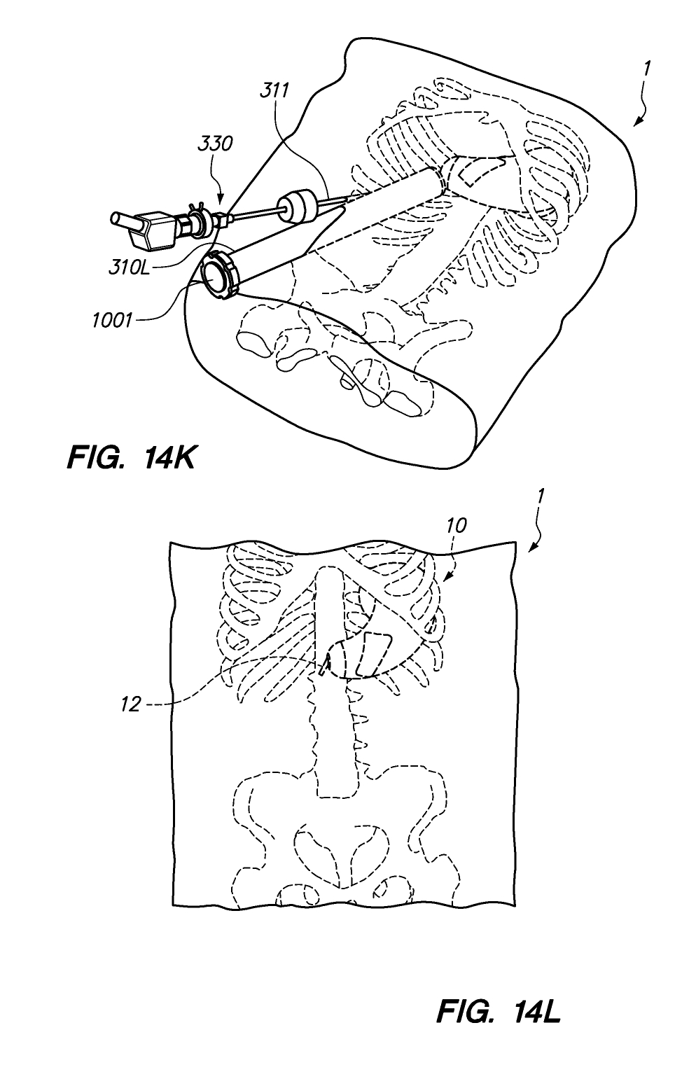

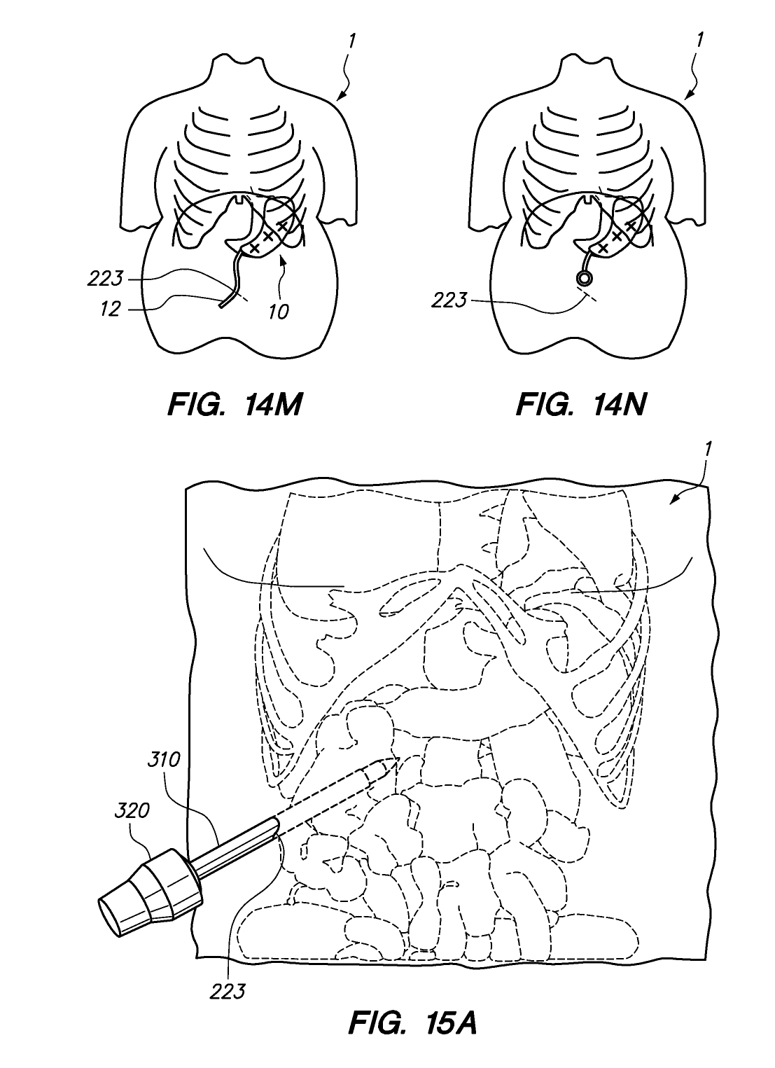

FIGS. 14A-14N illustrate an example of a procedure for implanting an extra-gastric, paragastric device 10 according to an embodiment of the present invention. The attachment tool 4000, suturing/stitching tool 5000, introducer cannula 310L, dilator 570 and endoscope 330 are not limited to the type of procedure described with regard to FIGS. 14A-14N, but this procedure is described in detail to facilitate a detailed understanding of the present invention, including use of these instruments and devices. After preparing the patient 1 for surgery, an incision or puncture 223 is made and an optical trocar/cannula 320/310 with an endoscope 330 inserted therein (e.g., a VISIPORT™ trocar with VERSAPORT™ PLUS trocar sleeve from Covidien may be used, or an OPTIVIEW® trocar from Ethicon Endosurgery, Inc. may be used, and a 10 mm endoscope may be used, wherein the shaft of the endoscope has 10 mm outside diameter) are inserted into the incision and advanced under visualization by endoscope 330 and/or by fluoroscopic visualization to enter the peritoneal cavity.

In this embodiment, incision 223 is made midline at a predetermined distance inferior of the xiphoid process. For example, the distance below the xiphoid process may be about 15 cm, although this distance may vary depending upon a number of factors, including, but not limited to the size of the patient and the body mass index of the patient. Alternatively, the incision 223 may be made at a predetermined distance (e.g., about 15 cm) inferior of the xiphoid process and at a predetermined distance (e.g., about 6 cm) to the patient's right of midline. FIG. 63A of application Ser. No. 12/474,226 illustrates an example of placement of the incision 223 to the right of midline. Initially, the trocar 320, cannula 310 and endoscope 330 are inserted into incision 223 at a substantially perpendicular orientation to the surface of the skin 125, as schematically illustrated in FIG. 14A. FIG. 14B illustrates the placement of a second port/cannula 311 into incision 223, which placement may be facilitated by a second trocar 321. The second port/cannula 311 is smaller than the first port/cannula 310. In at least one embodiment, the second port/cannula has an inside diameter of about five mm.

Once the sharpened tip of the trocar 320 has passed through the fascia/abdominal muscle of the patient 1 and it and the distal tip of the cannula 310 have entered the abdominal cavity, the distal tip of the second cannula 311 (delivery of which may be facilitated by a sharpened tip of trocar 321) enter the abdominal/peritoneal cavity through the same opening through the skin, but a different opening through the fascia/abdominal muscle, alongside cannula 311. Next, the trajectories of the cannulae 310, 311 trocars 320 (and optionally, 321) and endoscope 330 are flattened relative to the skin 125 of the patient 1 surrounding the incision 223, as schematically illustrated in FIG. 14C. In FIG. 14C, trocar 320 has been removed and endoscope 330 has been inserted back into the cannula 310. Trocar 321 has been removed from cannula 311 in order to allow the working end of any tools inserted therethrough to extend beyond the distal end of the cannula 311. The flattening of the trajectory angle forms an angle relative to the original, perpendicular orientation of greater than about 60 degrees, typically greater than about 80 degrees, and, in some embodiments, 90 degrees or more. Before or after angling the cannulae 310,311 as described above, but after the distal ends thereof have entered the abdominal/peritoneal cavity the trocar 320 and endoscope 330 can be removed from the cannula 310 and the endoscope 330 can then be reinserted into cannula 310. If a trocar 321 was used, it can be removed from the cannula 311 at this time.

By viewing provided through the endoscope 330, the surgeon can locate the falciform ligament and visually determine whether it is obscuring or attaching to the “landing zone”, where the term “landing zone” refers to the location where ingrowth material of the attachment tab 150 will contact tissue for attachment thereto and ingrowth thereby. If it is determined that the falciform ligament is obstructing or attached to the landing zone, then an instrument 370 can be inserted through cannula 311 and the working end of the instrument 370 can be extended out of the distal end of cannula 311 and manipulated to remove a portion of the falciform ligament that is obstructing the landing zone and/or a pathway along which the implant 10 is to be delivered. In FIG. 14C, the instrument 370 that is being used is a cauterizing grasper. Alternatively, cauterizing scissors may be used, or endoscopic scissors, or other alternative endoscopic tool sized to be inserted through cannula 311 and configured to perform the cutting operations required. It is preferable that the tool cauterizes as well as cuts or ablates.

Once there is a pathway toward the surgical target (implantation site) clear of the falciform ligament, the cannula 310 and endoscope 330 are removed from the patient 1 and the same or a different endoscope 330 (e.g., in the case where a relatively larger endoscope was used in cannula 310 and a relatively smaller endoscope 330 is needed to fit within cannula 311) is inserted into cannula 311 (after having removed any instruments that may be present in cannula 311, such as instrument 370). The dilator/introducer cannula assembly 570/310L are then inserted through opening 223 and through the opening in the fascia, while visually monitoring the advancement of the assembly 570/301L via visualization provided through endoscope 330 inserted through cannula 311 and/or by fluoroscopic visualization, and while providing insufflation to the abdominal cavity according to standard laparoscopic procedure used by surgeons, as schematically illustrated in FIG. 14D.

The dilator/introducer assembly 570/310L is advanced, while maintaining insufflation of the abdominal cavity and with visual monitoring via endoscope 330 and/or by fluoroscopic visualization, to a location where the distal tip 570d of the dilator 570 touches or nearly touches (approximates) the approximate target location where the implant device 10 is to be placed (i.e., the diaphragm 116 of the patient 1, as illustrated in FIG. 14E). This positions the distal end of the introducer cannula 310L appropriately for placement of the device 10 in the vicinity of the landing zone, roughly in the appropriate location for implantation.

When the distal tip of the dilator 570 has been positioned as desired as shown in FIG. 14E, the dilator 570 is next decoupled and removed from introducer cannula 310L, while maintaining the introducer cannula 310L fixed in the position established in the prior step (FIGS. 14E-14F). Once the dilator has been removed (FIG. 14F), the insufflation pressure is eliminated or greatly reduced due to the outflow of insufflation fluid/gas through the annulus of the large cannula which is now open at the proximal end, as illustrated in FIG. 14F.

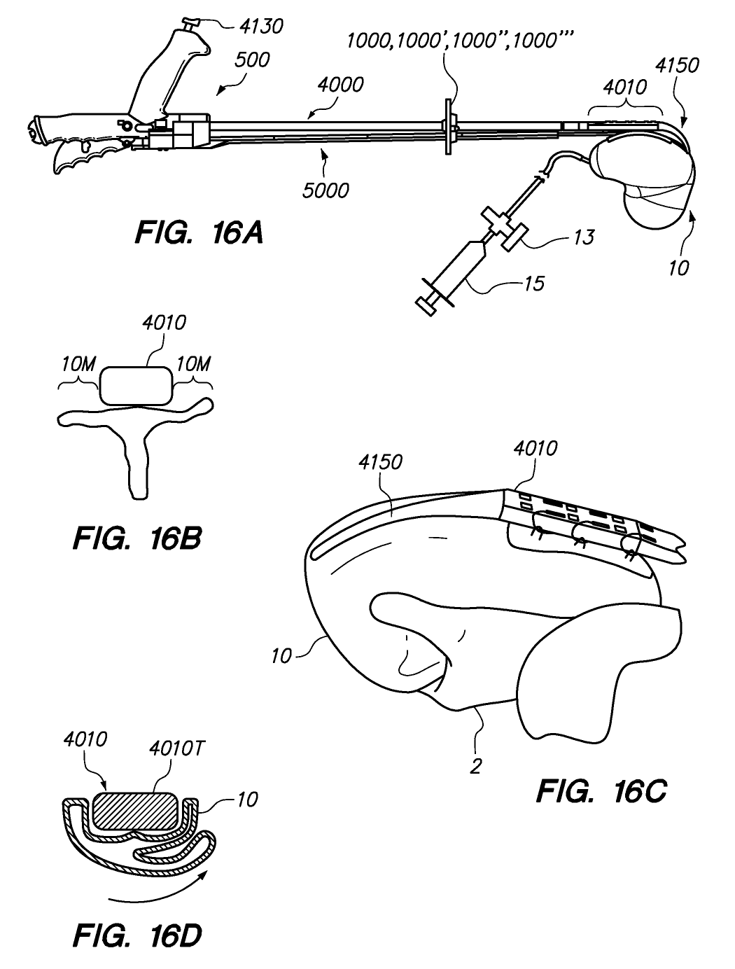

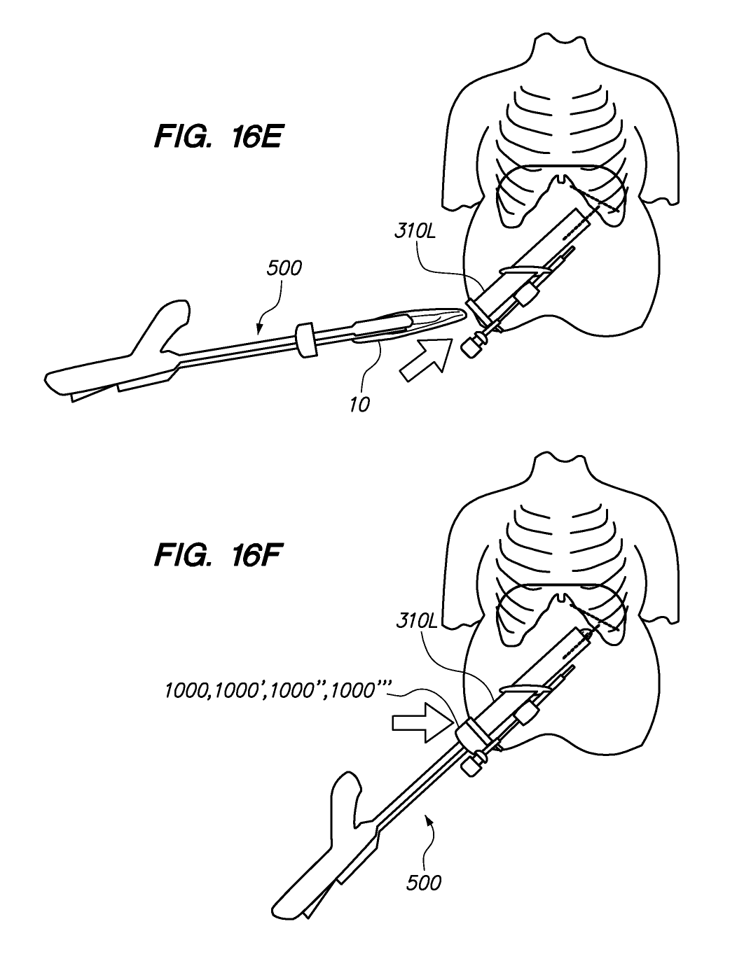

Prior to this, the assembly 500 will have been prepared for use (an embodiment of such preparation is described in detail below with regard to FIGS. 16A-16F, having a sealing member (1000 as shown; alternatively 1000′, 1000″ or 1000′″ may be substituted) provided over shafts 4140, 5140 and having device 10 mounted thereto in a compact (non-enlarged) configuration, as illustrated in FIG. 14F. Although reference numeral 1000 has been used in FIG. 14F and throughout the FIG. 14 series to denote the sealing member, it is noted that this procedure is not limited to sealing member 1000, as any of the other variants of sealing member (e.g., 1000′, 1000″, 1000′″) described herein could be substituted. Likewise, other introducers/cannulae could be substituted for cannula 310L. The distal end of assembly 500, including implant 10 are then inserted into the introducer cannula 310L as indicated by the arrow in FIG. 14F.

Once the implant 10 has been fully inserted into the introducer cannula 310L and the proximal end thereof is distal of the proximal end of the introducer cannula/handle 310L/590h by at least the length to the sealing member 1000, the sealing member 1000 is advanced distally and attached to the proximal end/handle of the introducer cannula 310L/590h in a manner as described above or below herein. This seals off the proximal end of the introducer cannula 310L and allows insufflation pressure to be reestablished in the abdominal cavity. Once full insufflation pressure has been achieved (or substantially achieved), assembly 500 is advanced distally while maintaining the position of introducer cannula 310L. The assembly 500 is advanced until the implantable device 10 contacts or nearly contacts (approximates) the approximate target location where the implant device 10 is to be implanted (i.e., the diaphragm 116, as illustrated in FIG. 14G). This can be visually confirmed by visualizations obtained through endoscope 330 and/or by fluoroscopic visualization.

Next, the introducer cannula 310L is retracted proximally while maintaining the position of the device 10 and assembly 500 as illustrated in FIG. 14H. Insufflation pressure is maintained during this step, and the retraction of the cannula 310L can be visually monitored through endoscope 330. Cannula 310L is retracted until at least the enlargeable portion 10em of the device 10 is fully exposed (i.e., extends distally of the distal end of cannula 310L), as shown in FIG. 14H.

Alternatively, the cannula 310L can be made shorter than in the above embodiment, so that retraction thereof is not necessary. In this alternative embodiment the device 10 and assembly 500 are simply advanced relative to cannula 310L until at least the enlargeable portion 10em of the device 10 is fully exposed (i.e., extends distally of the distal end of cannula 310L), without the need to retract the cannula 310L.

Next the implantable device 10 is enlarged from its compact configuration to an enlarged configuration, as illustrated in FIG. 14I. In the embodiment shown, the device 10 is enlarged by filling it with fluid (e.g., saline) through filling tube 12. Although filling tube 12 is shown only schematically in FIG. 14I, in actuality it extends further proximally from the enlargeable member 10em so as to extend out of the patient's body 1, where it can be connected with a pressurized fluid source. Preferably, the device 10 is filled until the top of the device 10 contacts the patient's diaphragm, or until it has reached the volume that has been predetermined to be appropriate for the patient (through the use of MRI imaging and/or the template assessment at the beginning of the procedure). Further details about filling tube 12 can be found in application Ser. Nos. 12/474,226; 12/473,818; 12/474,118; 11/716,986; 11/716,985; and 11/407,701. It is further noted, that although the device 10 embodiment shown in this example is a tillable or inflatable device 10, that the present invention is not limited to this type of device as other types of enlargeable devices could be substituted, such as a mechanically enlargeable device, a hybrid device that includes both mechanical and fillable enlargement features, etc.

While still under full insufflation, the positioning of the enlarged device 10 is visually inspected through endoscope 330. During this inspection, careful attention is paid to the location and orientation of the attachment tab 150 and to ensure that no obstructions or other tissues are located between the attachment tab and the attachment site (abdominal wall, fascia). The placement and orientation of the enlarged member 10em are also noted. If repositioning is needed, device 10 can be reduced in size by partial up to total deflation and assembly 500 can be manipulated to reposition the implant, after which it is enlarged again to the state shown in FIG. 14I. This process can be iterated as many times as necessary to establish satisfactory placement and orientation of the device 10 and attachment tab 150. Once satisfactory placement and orientation has been achieved, insufflation pressure is reduced by an amount according to the surgeon's choice, typically being reduced to a level that is about one half to about three-quarters of the previous pressure, or reduce to zero insufflation pressure, or anywhere in between, while maintaining device 10 in the enlarged configuration shown in FIG. 14I.

Optionally, an endoscope 330 may be inserted into a left side lumen (not shown, see application Ser. No. 12/474,226 for details) that extends from a proximal end portion of instrument 4000 to a location just proximal of working end portion 4010 and to a location alongside of the working end portion 4010, and endoscope 330 is used to view between the abdominal wall (e.g., fascia/peritoneum) and the working end portion 4010 to ensure that no omentum, bowel or other organs or tissues are in the pathway along which the stitching needles are to be driven into and out of the fascia/peritoneum, abdominal wall.

In one embodiment, when it has been determined that the pathways for the stitching needles on the left side of the working end portion 4010 are clear to be advanced, then the endoscope 330 is removed from left side lumen and inserted into a lumen on the right side of the instrument (also shown and described in application Ser. No. 12/474,226). The right side lumen extends from a proximal end portion of instrument 4000 to a location just proximal Of working end portion 4010 and alongside of working end portion 4010, such that endoscope, when inserted therein, is used to view between the abdominal wall 127 (e.g., fascia/peritoneum 127f) and the working end portion 4010 to ensure that no omentum, bowel or other organs or tissues are in the pathway along which the stitching needles on the right side of the working end portion 4010 are to be driven into and out of the fascia/peritoneum, abdominal wall. Thus, endoscopic visualization via endoscope 330 through cannula 311 and/or the left and right lumens along the sides of tool 4000, is used to confirm that the attachment location is clear of omentum, bowel, etc., e.g., that the tool 4000 and portion of the device 10 to be attached are positioned so that a clear pathway to the attachment site exists, such that no bowel, excessive fat, or other obstruction exists between the attachment tab 150 and the attachment location, such as the abdominal wall, costal cartilage, or other internal body structure to which device 10 is to be attached.

In another embodiment, the direct delivery allows the endoscope 330 to be inserted through cannula 311 to be manipulated to provide a view above the end effector to assess both sides.

When the “landing zone” has been visually confirmed as being clear, a local anesthetic, such as Lidocaine, Marcaine, or the like can be delivered to the target implantation site (e.g., at least one of fascia, peritoneum, preperitoneal fat and/or posterior rectus sheath) through a lumen in tool 4000, such as through one of lumens used to insert the endoscope for viewing, after removal of the endoscope, for example, or by needle and syringe, trans-abdominally. Attachment tool 4000 is next actuated to perform the initial attachment of device 10 to the patient's body, and to thereby anchor the sutures 444 to suture anchors or traps as described in application Ser. No. 12/474,226. Light counter pressure can be applied to the patient on the skin over the landing zone and/or the distal end of tool 4000 can be raised up against the inside of the patient to help ensure that the stitching needles can penetrate easier and as deep as possible into the tissue. Although the attachment tool, as described in application Ser. No. 12/474,226 and as used herein preferably rotates the stitching needles toward a distal end of the tool 4000, an alternative embodiment can be used wherein the stitching needles are rotated toward the proximal end of the tool. After completion of this initial attachment/stitching, tool 4000 is separated from tool 5000 and removed from the patient 1 and out of the introducer cannula 310L. Once completely removed, the first valve 1012 (or 1012′, 1012″ or stopper 1012′″) is automatically or manually closed so that sealing member 1000 maintains the sealing off of the proximal end of introducer cannula (FIG. 14J) to substantially maintain the current level of insufflation or at least to allow any insufflation pressure lost during removal of the tool 4000 to be quickly reinstated.

Next, the sutures are cinched in the direction of the arrow in FIG. 14J (sutures not shown in FIG. 14J, but shown and described in detail in application Ser. No. 12/474,226), secured by suture retainers (not shown) and the excess proximal portions of the sutures 444 are cut off. This process can be visually monitored by visualization through cannula 311 using endoscope 330, as illustrated in FIG. 14J, and insufflation pressure is maintained as facilitated by sealing member 1000.

The suturing instrument 5000 is then removed from the patient 1, leaving the introducer cannula 310L and cannula/endoscope 311/330. The sealing member 1000 remains attached to the cannula 310L. Next, a cap 1001 is attached to the proximal end of the introducer cannula 310L, as shown in FIG. 14K, to seal it off. Alternatively, when sealing member 1000′″ is used, the ports 1008′″ and 1010′″ are sealed off using plugs 1044 and 1054, respectively (FIGS. 6B and 6A) and cap 1001 is not needed. Next, under full insufflation, or a lesser level of insufflation pressure, according to the surgeon's choice, the attachment of the attachment tab to the tissues is inspected, using the endoscope 330 inserted through the cannula 311.

Once it has been determined that the attachment of the attachment tab 150 and thus the device 10 has been performed satisfactorily, the cannula 310L and cannula 311/endoscope 330 are removed from the patient leaving only the implanted device 10 in the patient 1 (FIG. 14L) and allowing the abdominal cavity to desufflate.

Filling tube 12 extends proximally out of opening 223, as illustrated in FIG. 14M. At FIG. 14N, filling tube 12 is cut to the appropriate length to join adjustment member 80 thereto and to reduce any excessive length of filling tube 12 that might otherwise exist. After securing adjustment member 80 to the fascia/abdominal wall to both anchor it as well as to close the opening through the fascia, any adjustment of the volume of expandable member 10em can be performed as needed, and then the patient can be closed, including closing of opening 223 to complete the procedure. Adjustment member 80 can be installed/attached to the abdominal wall/fascia at a location other than the opening 223. In such cases, opening 223 is closed around the fill tube 12 extending therefrom, and the adjustment member 80 is attached to the fascia and/or abdominal muscle at another location, so that attachment member 80 does not need to perform the closure function for closing the opening 223. Further details of this and other procedures that can be performed with the devices of the present invention are described in application Ser. No. 61/130,244, which is hereby incorporated herein, in its entirety, by reference thereto, and in co-pending application Ser. Nos. 12/474,226; 12/473,818; and 12/474,118.

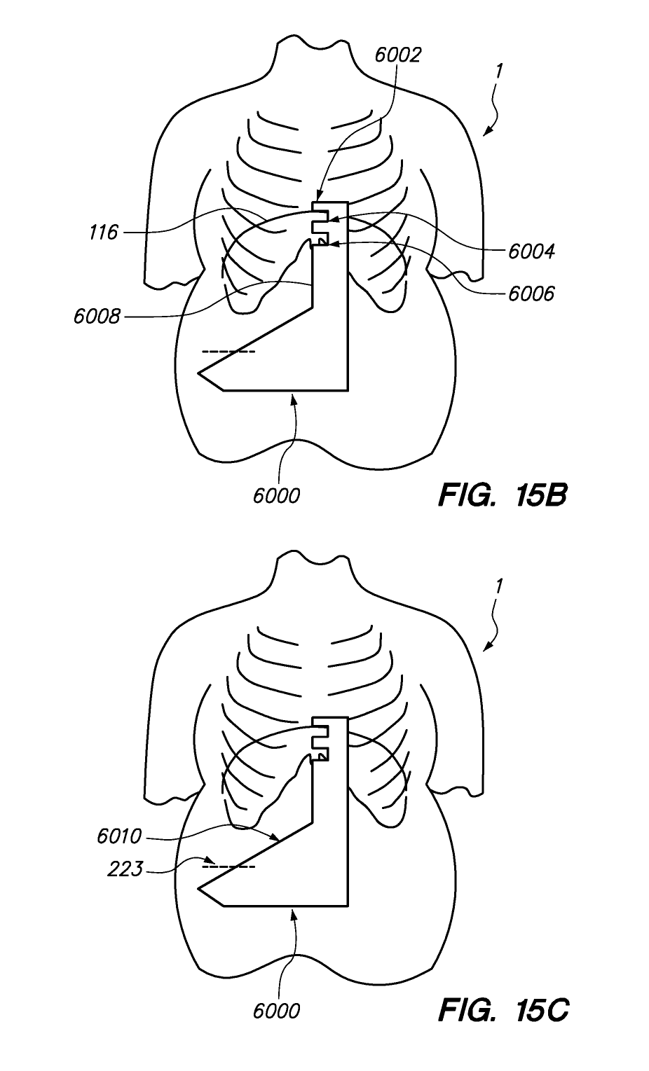

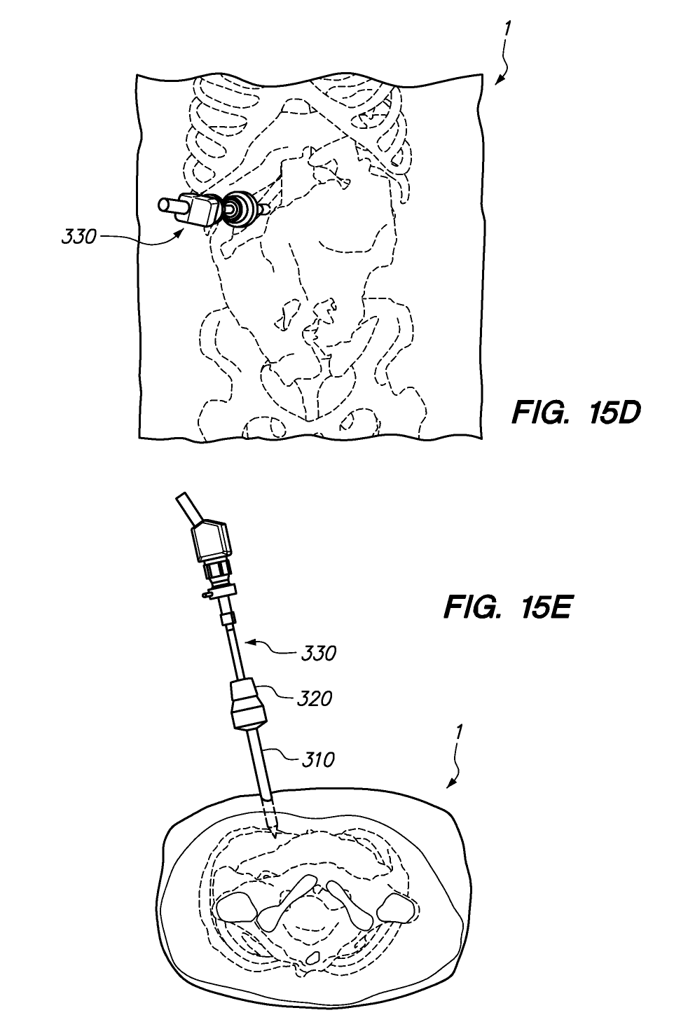

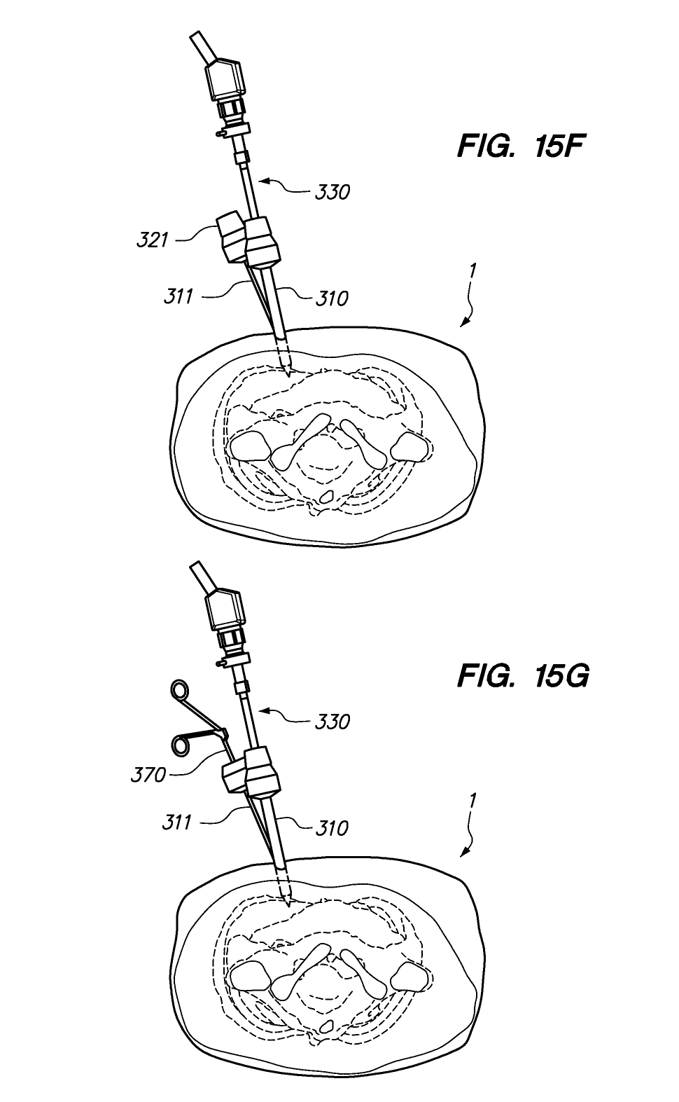

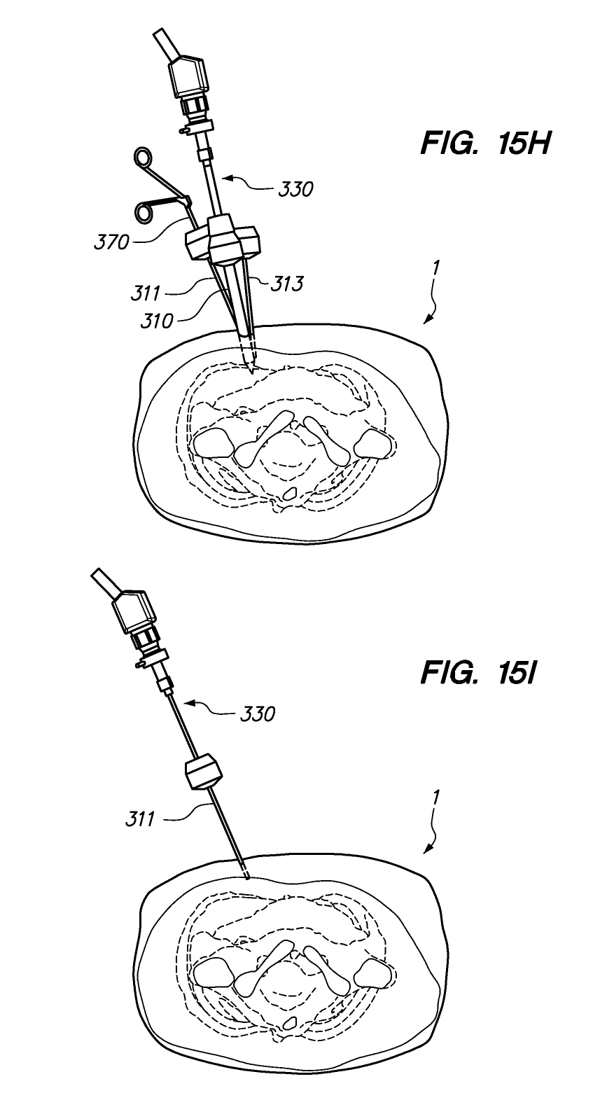

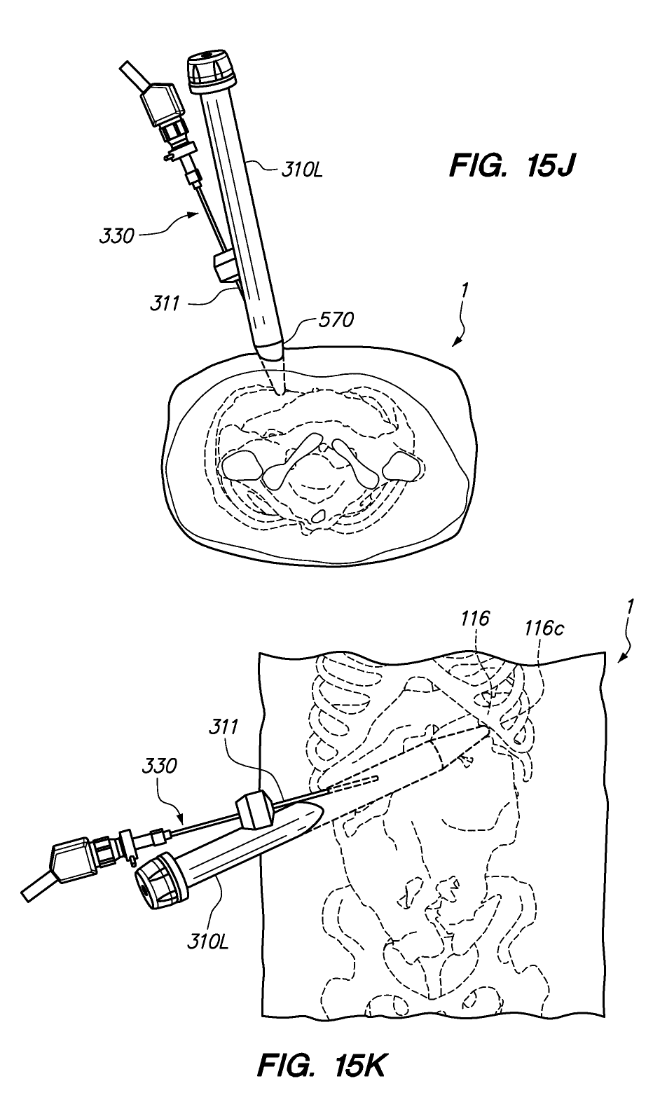

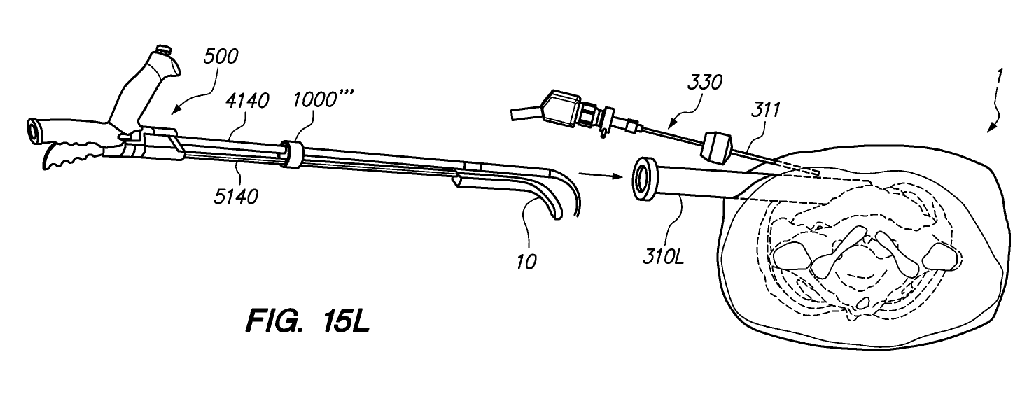

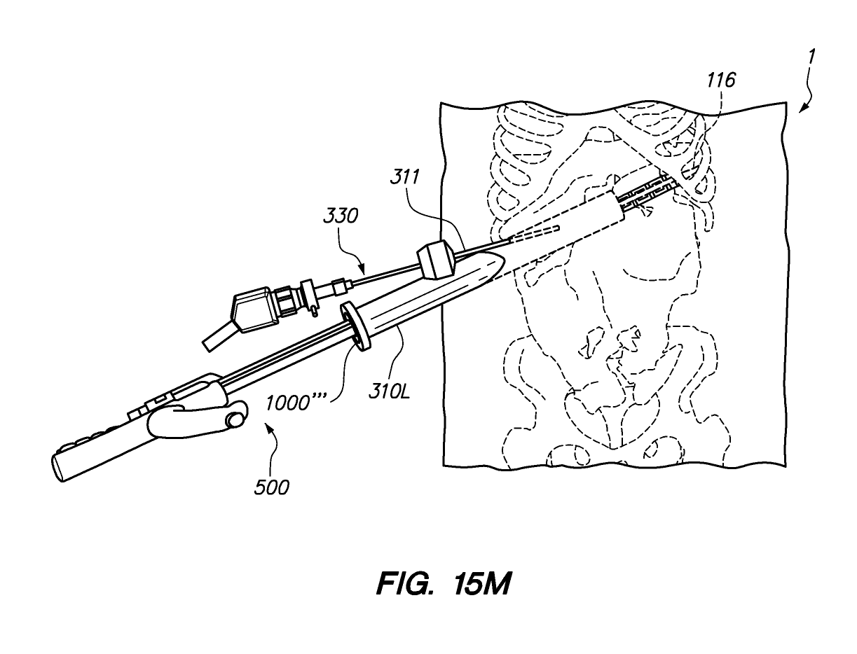

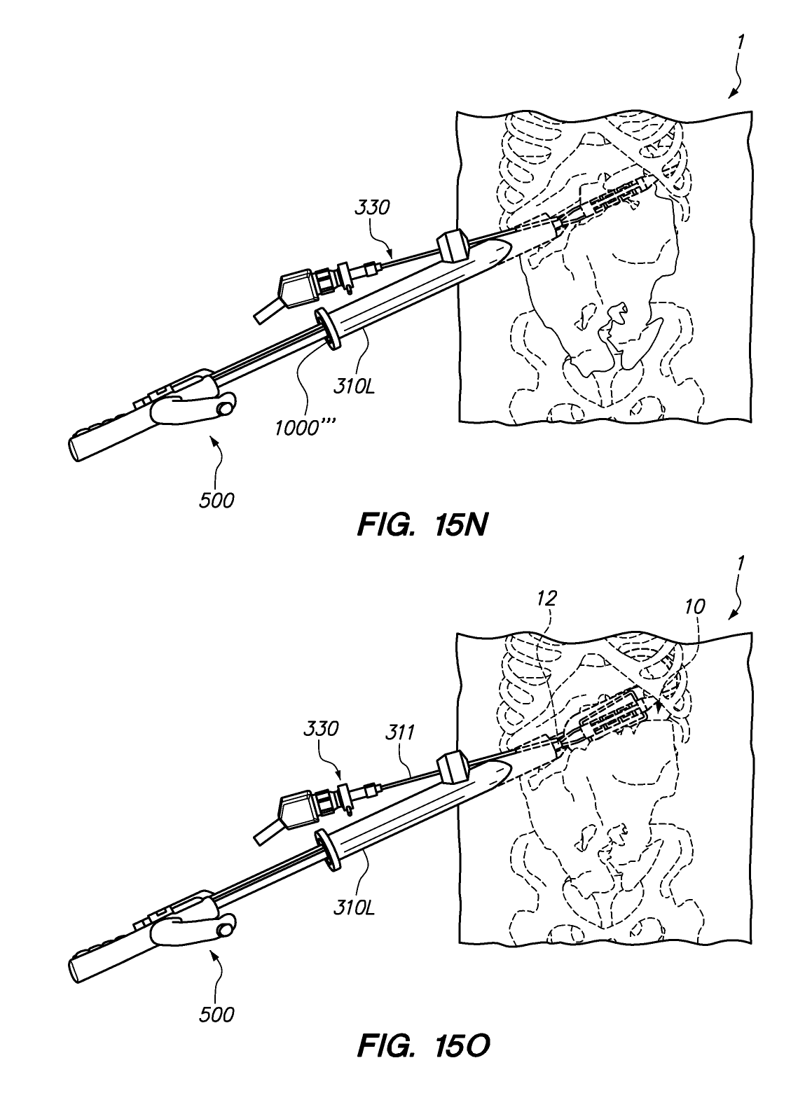

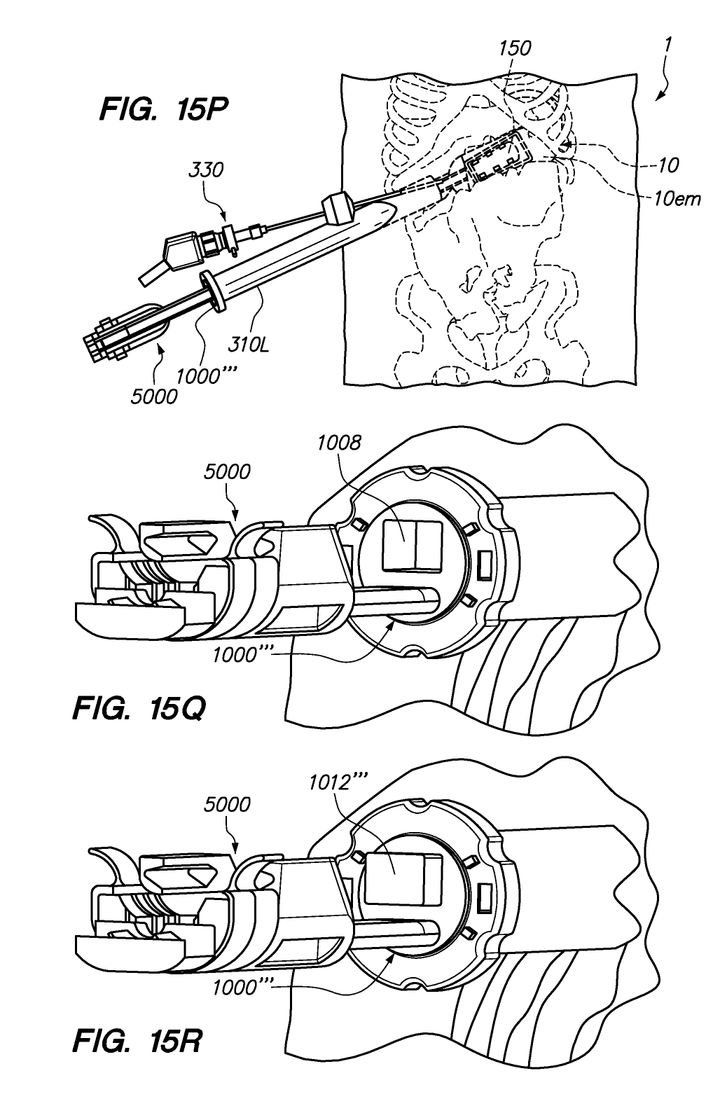

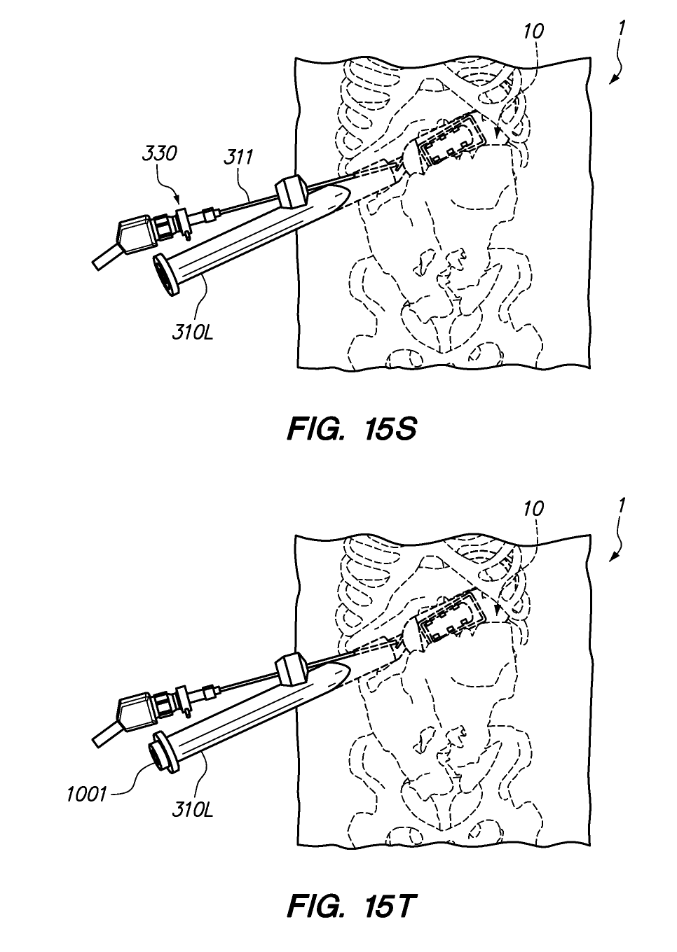

FIGS. 15A-15T illustrate an example of a procedure for implanting an extra-gastric, paragastric device 10 according to another embodiment of the present invention. This embodiment is substantially similar to the embodiment described above with regard to FIGS. 14A-14N, except that the incision or puncture 223 is made inferior of the xiphoid process and to the right (patient's right) of midline. After preparing the patient 1 for surgery, an incision 223 is made and an optical trocar/cannula 320/310 with an endoscope 330 inserted therein are inserted into the incision and advanced under visualization by endoscope 330 (see FIG. 15A). Insufflation is applied via a standard, laparoscopic trocar port.

Optionally, a template is used to determine the incision location, as illustrated in FIGS. 15B-15C. In this embodiment, incision or puncture 223 is made at a predetermined distance inferior of the xiphoid process and a predetermined distance to the right of midline of the patient 1, see FIG. 15A. For example, the distance below the xiphoid process may be about 15 cm and the distance to the right of midline may be about 6 cm, although these distances may vary. Initially, the trocar 320, cannula 310 and endoscope 330 are inserted into incision 223 at a substantially perpendicular orientation to the surface of the skin 125. Once the sharpened tip of the trocar 320 has passed through the fascia/abdominal muscle and it and the distal tip of the cannula 310 have entered the abdominal cavity, the trajectory of the cannula 310, trocar 320 and endoscope 330 is flattened relative to the skin of the patient surrounding the incision/puncture 223 to form an angle 331 relative to the original, perpendicular orientation of greater than about 60 degrees, typically greater than about 80 degrees, and, in some embodiments, 90 degrees or more.

Optionally, as illustrated in FIGS. 15B-15C, a positioning template 6000 may be used to locate where, on the patient's 1 abdomen, to make the incision or puncture 223. At FIG. 15B, after using fluoroscopy and a radiopaque marker to mark the approximate level of the diaphragm 116 on the skin, as identified using the fluoroscopy, the positioning template 6000 is placed on the patient 1 with the top portion aligned with the diaphragm 116 according to which implant 10 size is to be used (see application Ser. No. 12/474,226 for a detailed description of the provision of implants of various sizes from which a selection can be made). For example, in FIG. 15B, the top edge 6002 of the template 6000 is aligned with the diaphragm 116 when the largest available device 10/enlargeable member 10em is to be used (e.g., “implant size F”). In the example shown in FIG. 15B, the user is planning to implant the next smaller size device 10/enlargeable member 10em (e.g., “implant size E”) and therefore the notch at 6004 has been aligned with the marking that indicates the level of the diaphragm 116. An additional notch 6006 is provided below notch 6004 for use when a yet smaller sized implant is to be implanted (e.g., implant size B, C or D). Additionally, the template is adjusted so that the left vertical edge 6008 of template 6000 is substantially aligned with the patient's spine.

Next, using the marking pen a line is drawn on the patient's abdomen along the trajectory edge 6010 of the template as indicated in FIG. 15C to indicate the intended trajectory for placement of the assembly 500. The center of the abdominal incision/puncture 223 should be made where the line formed along 6010 crosses the right linea semiluminaris. A short-action local anesthetic (e.g., Lidocaine or the like) can be applied prior to making the incision/puncture 223. Incision/puncture 223 is made to have a length/radius of approximately 5 cm in the location shown in FIG. 15C. Once the incision/puncture 223 is made, the procedure continues as described above with regard to FIGS. 15A, 15D and 15E.

FIG. 15F illustrates the placement of a second port/cannula 311 into incision/puncture 223, which placement may be facilitated by a second trocar 321. The second port/cannula 311 is smaller than the first port/cannula 310. In at least one embodiment, the second port/cannula has an inside diameter of about five mm.

Once the sharpened tip of the trocar 320 has passed through the fascia/abdominal muscle of the patient 1 and it and the distal tip of the cannula 310 has entered the abdominal cavity, the distal tip of the second cannula 311 (delivery of which may be facilitated by a sharpened tip of trocar 321) enters the abdominal/peritoneal cavity through the same opening through the fascia/abdominal muscle, alongside cannula 311. By viewing provided through the endoscope 330, the surgeon can locate the falciform ligament and visually determine whether it is obscuring or attaching to the “landing zone”. If it is determined that the falciform ligament is obstructing or attached to the landing zone, then an instrument 370 can be inserted through cannula 311 and the working end of the instrument 370 can be extended out of the distal end of cannula 311 and manipulated to remove a portion of the falciform ligament that is obstructing the landing zone and/or a pathway along which the implant 10 is to be delivered. In FIG. 150, the instrument 370 that is being used is a cauterizing grasper. Alternatively, cauterizing scissors may be used, or endoscopic scissors, or other alternative endoscopic tool sized to be inserted through cannula 311 and configured to perform the cutting operations required. It is preferable that the tool cauterizes as well as cuts or ablates.

Optionally a third cannula/port 313 may be inserted through the incision/puncture 223 as illustrated in FIG. 15H, to allow additional instrumentation, such as graspers, endoscope, electrocautery tool, or other instrument, to be inserted therethrough. Third cannula/port 313 is typically of the same size as second cannula/port 311, but need not be. Once there is a pathway toward the surgical target (implantation site) clear of the falciform ligament, the landing zone can be marked (such as by electrocautery). The cannula 310 and endoscope 330 are removed from the patient 1, the tool 370 is removed from cannula 311, and the same or a different endoscope 330 (e.g., in the case where a relatively larger endoscope was used in cannula 310 and a relatively smaller endoscope 330 is needed to fit within cannula 311) is inserted into cannula 311 (after having removed any instruments that may be present in cannula 311, such as instrument 370), as illustrated in FIG. 15I.

The dilator/introducer cannula assembly 570/310L are then inserted through opening 223 and through the opening in the fascia, while visually monitoring the advancement of the assembly 570/301L via visualization provided through endoscope 330 inserted through cannula 311 and/or by fluoroscopic visualization, and while providing insufflation to the abdominal cavity, in an amount according to the surgeon's choice, such as typical in standard laparoscopic procedures, FIG. 15J.

The dilator/introducer assembly 570/310L is advanced, while maintaining insufflation of the abdominal cavity and with visual monitoring via endoscope 330 and/or by fluoroscopic visualization, to a location where the distal tip 570d of the dilator 570 touches or nearly touches (approximates) the approximate target location where the implant device 10 is to be placed (e.g., the diaphragm 116 or costal margin 116c of the patient 1, as illustrated in FIG. 15K). This positions the distal end of the introducer cannula 310L appropriately for placement of the device 10 in the vicinity of the landing zone roughly in the appropriate location for implantation.

When the distal tip of the dilator 570 has been positioned as desired as shown in FIG. 15K, the dilator 570 is next decoupled and removed from introducer cannula 310L, while maintaining the introducer cannula 310L fixed in the position established in the prior step. Once the dilator has been removed (FIG. 15L), the insufflation pressure is eliminated or greatly reduced due to the outflow of insufflation fluid/gas through the annulus of the large cannula 310L which is now open at the proximal end, as illustrated in FIG. 15L.

Prior to this, the assembly 500 will have been prepared for use, (an embodiment of such preparation is described in detail below with regard to FIGS. 16A-16F, having a sealing member (1000′″ as shown; alternatively 1000, 1000′ or 1000″ may be substituted) provided over shafts 4140, 5140 and having device 10 mounted thereto in a compact (non-enlarged) configuration, as illustrated in FIG. 15L. Although reference numeral 1000′″ has been used in FIG. 15L and throughout the FIG. 10 series to denote the sealing member, it is noted that this procedure is not limited to sealing member 1000′″, as any of the other variants of sealing member (e.g., 1000, 1000′, 1000″) described herein could be substituted. The distal end of assembly 500, including implant 10 are then inserted into the introducer cannula 310L as indicated by the arrow in FIG. 15L.

Once the implant 10 has been fully inserted into the introducer cannula 310L and the proximal end thereof is distal of the proximal end of the introducer cannula/handle 310L/590h by at least the length of the sealing member 1000′″, the sealing member 1000′″ is advanced distally and attached to the proximal end/handle of the introducer cannula 310L/590h in a manner as described above or by forming a simple friction fit in the way that a stopper forms a friction fit with a flask. This seals off the proximal end of the introducer cannula 310L and allows insufflation pressure to be reestablished in the abdominal cavity. Once full insufflation pressure has been achieved (or substantially achieved), assembly 500 is advanced distally while maintaining the position of introducer cannula 310L. The assembly 500 is advanced until the implantable device 10 contacts or nearly contacts (approximates) the approximate target location where the implant device 10 is to be implanted (i.e., the diaphragm 116, as illustrated in FIG. 15M). This can be visually confirmed by visualizations obtained through endoscope 330 and/or by fluoroscopic visualization.

Next, the introducer cannula 310L is retracted proximally while maintaining the position of the device 10 and assembly 500 as illustrated in FIG. 15N. The sealing member 1000′″ slides along the shafts 4140, 5140 of assembly 500 as introducer cannula 310L is retracted relative to assembly 500, thereby maintaining insufflation pressure in the abdominal cavity. Thus, insufflation pressure is maintained during this step, and the retraction of the cannula 310L can be visually monitored through endoscope 330 and/or via fluoroscopy. Cannula 310L is retracted until at least the enlargeable portion 10em of the device 10 is fully exposed (i.e., extends distally of the distal end of cannula 310L), as shown in FIG. 15N.

Next the implantable device 10 is enlarged from its compact configuration to an enlarged configuration, as illustrated in FIG. 15O. In the embodiment shown, the device 10 is enlarged by filling it with fluid (e.g., saline) through filling tube 12. Although filling tube 12 is shown only schematically in FIG. 15O, in actuality it extends further proximally from the enlargeable member 10em so as to extend out of the patient's body 1, where it can be connected with a pressurized fluid source. Further details about filling tube 12 can be found in application Ser. Nos. 12/474,226; 12/473,818; 12/474,118; 11/716,986; 11/716,985; and 11/407,701. It is further noted, that although the device 10 embodiment shown in this example is a finable or inflatable device 10, that the present invention is not limited to this type of device as other types of enlargeable devices could be substituted, such as a mechanically enlargeable device, a hybrid device that includes both mechanical and fillable enlargement features, etc.

While still under full insufflation, the positioning of the enlarged device 10 is visually inspected through endoscope 330. During this inspection, careful attention is paid to the location and orientation of the attachment tab 150 and to ensure that no obstructions or other tissues are located between the attachment tab and the attachment site (abdominal wall, fascia). The placement and orientation of the enlarged member 10em are also noted. If repositioning is needed, device 10 can be reduced in size by partial up to nearly total deflation and assembly 500 can be manipulated to reposition the implant, after which it is enlarged again to the state shown in FIG. 15O. This process can be iterated as many times as necessary to establish satisfactory placement and orientation of the device 10 and attachment tab 150. Once satisfactory placement and orientation has been achieved, insufflation pressure is reduced by an amount according to the surgeon's choice, typically being reduced to a level that is about one half to about three-quarters of the previous pressure, or reduced to zero insufflation pressure, or anywhere in between (in one example pressure is reduced from about 15 mmHg to about 3 mmHg), while maintaining device 10 in the enlarged configuration shown in FIG. 15O.

Optionally, an endoscope 330 may be inserted into a left side lumen (not shown, see application Ser. No. 12/474,226 for details) that extends from a proximal end portion of instrument 4000 to a location just proximal of working end portion 4010 and to a location alongside of the working end portion 4010, and endoscope 330 is used to view between the abdominal wall (e.g., fascia/peritoneum) and the working end portion 4010 to ensure that no omentum, bowel or other organs or tissues are in the pathway along which the stitching needles are to be driven into and out of the fascia/peritoneum, abdominal wall. When it has been determined that the pathways for the stitching needles on the left side of the working end portion 4010 are clear to be advanced, then the endoscope 330 is removed from left side lumen and inserted into a lumen on the right side of the instrument (also shown and described in application Ser. No. 12/474,226). The right side lumen extends from a proximal end portion of instrument 4000 to a location just proximal of working end portion 4010 and alongside of working end portion 4010, such that endoscope, when inserted therein, is used to view between the abdominal wall (e.g., fascia/peritoneum) and the working end portion 4010 to ensure that no omentum, bowel or other organs or tissues are in the pathway along which the stitching needles on the right side of the working end portion 4010 are to be driven into and out of the fascia/peritoneum, abdominal wall. Thus, endoscopic visualization via endoscope 330 through cannula 311 and/or the left and right lumens along the sides of tool 4000, is used to confirm that the attachment location is clear of omentum, bowel, etc., e.g., that the tool 4000 and portion of the device 10 to be attached are positioned so that a clear pathway to the attachment site exists, such that no bowel, excessive fat, or other obstruction exists between the attachment tab 150 and the attachment location, such as the abdominal wall, costal cartilage, or other internal body structure to which device 10 is to be attached.

As an alternative option, the direct delivery allows the endoscope 330 to be inserted through cannula 311 to be manipulated to provide a view above the end effector to assess both sides.

When the landing zone has been visually confirmed as being clear, a local anesthetic, such as Lidocaine, Marcaine, or the like can be delivered to the target implantation site (e.g., the fascia/peritoneum and abdominal wall) through a lumen in tool 4000, such as through one of lumens used to insert the endoscope for viewing, after removal of the endoscope, for example. Attachment tool 4000 is next actuated to perform the initial attachment of device 10 to the patient's body, and to thereby anchor the sutures 444 to suture anchors or traps as described in application Ser. No. 12/474,226. Light counter pressure can be applied to the patient's skin over the landing zone and/or the distal end of the tool 4000 can be raised up against the inside of the patient to help ensure that the stitching needles can penetrate easier and as deep as possible into the tissues. After completion of this initial attachment/stitching, tool 4000 is separated from tool 5000 and removed from the patient 1 and out of the introducer cannula 310L, as illustrated in FIG. 15P. Once completely removed, the first port 1008 (see FIG. 15Q) of the sealing member 1000′″ is sealed off by inserting plug/stopper 1012′″ therein, as illustrated in FIG. 15R. Alternatively, if one of the other embodiments of sealing member 1000′″ is used, first valve 1012, 1012′ or 1012″ is automatically or manually closed. In any case, sealing member 1000′″ (or 1000, 1000′, or 1000″) then maintains the sealing off of the proximal end of introducer cannula (FIG. 15R) to substantially maintain the current level of insufflation or at least to allow any insufflation pressure lost during removal of the tool 4000 to be quickly reinstated.

Next, the sutures are cinched in the same manner as described above with regard to FIG. 14J and described in detail in application Ser. No. 12/474,226, secured by suture retainers (not shown) and the excess proximal portions of the sutures 444 are cut off. This process can be visually monitored by visualization through cannula 311 using endoscope 330, and insufflation pressure is maintained as facilitated by sealing member 1000′″.

The suturing instrument 5000 is then removed from the patient 1, leaving the introducer cannula 310L and cannula/endoscope 311/330, as illustrated in FIG. 15S. Sealing member 1000 remains attached to cannula 310L. Next, a cap 1001 is attached to the proximal end of the introducer cannula 310L, as shown in FIG. 15T (or, alternatively, plugs 1044 and 1054 are used to plug and seal the ports of the sealing member), to seal it off and full insufflation is reintroduced to the abdominal cavity. Next, under insufflation, the attachment of the attachment tab 150 to the tissues is inspected, using the endoscope 330 inserted through the cannula 311.

Once it has been determined that the attachment of the attachment tab 150 and thus the device 10 has been performed satisfactorily, the cannula 310L and cannula 311/endoscope 330 are removed from the patient leaving only the implanted device 10 in the patient 1 (like shown in FIG. 14L) and allowing the abdominal cavity to desufflate.

Filling tube 12 extends proximally out of opening 223, as illustrated in FIG. 14M (except that opening 23 is to the right of midline). Like described above with regard to FIG. 14N, the filling tube 12 is cut to the appropriate length to join adjustment member 80 thereto and to reduce any excessive length of filling tube 12 that might otherwise exist. After securing adjustment member 80 to the fascia/abdominal wall to both anchor it as well as to close the opening through the fascia, any adjustment of the volume of expandable member 10em can be performed as needed, and then the patient can be closed, including closing of opening 223 to complete the procedure. Adjustment member 80 can be installed/attached to the abdominal wall/fascia at a location other than the opening 223. In such cases, opening 223 is closed around the fill tube 12 extending therefrom, and the adjustment member 80 is attached to the fascia and/or abdominal muscle at another location, so that attachment member 80 does not need to perform the closure function for closing the opening 223. Further details of this and other procedures that can be performed with the devices of the present invention are described in application Ser. No. 61/130,244, which is hereby incorporated herein, in its entirety, by reference thereto, and in co-pending application Ser. Nos. 12/474,226; 12/473,818; and 12/474,118

FIGS. 16A-16F are now referred to by the following description of preparation of the assembly 500 and device 10 for use according to an embodiment of the present invention. FIG. 16A shows the device 10 connected to the distal end of the assembly 500. In a preferred embodiment, the assembly 500 and device 10 will be shipped to the end user in this configuration. Alternatively, when the assembly 500 and device 10 are received separately, then device 10 is attached to the assembly 500 at the commencement of preparation, and inflated (if not already inflated, although, typically, device 10 will be shipped in an inflated state to prevent creasing), as shown in FIG. 16A. The preparer ensures that the sealing member 1000, 1000′, 1000″, 1000′″ is positioned at a predetermined distance (e.g., about 6 cm, or some other predetermined distance found to be optimal) from the proximal end of working end portion/end effector 4010. If the sealing member 1000, 1000′, 1000″, 1000′″ is not positioned at the predetermined distance, than the preparer can slide it into the location where it is separated by the predetermined distance.

The device 10 is next immersed into sterile saline to check for leaks. If any leak is found, it needs to be replaced with a new device 10, or an entirely new assembly 500 and device 10 and retested. When no leaks are found, the leak free device is deflated by opening stopcock 13 and withdrawing fluid from the device 10 using a syringe 15, for example. While deflating the device 10, the user/preparer will ensure that the device 10 is flattened in a manner to minimize the amount of material of the device extending beyond the ends of the working end 4010, see 10M in FIG. 16B. When all or substantially all fluid has been removed from device 10, stopcock 13 is closed to maintain the deflated condition of device 10. FIG. 16C illustrates the hand 2 of the user/preparer manipulating the shape of device 10 as it is deflated, to ensure that no portion of the device extends above the end effector 4010 and so as to minimize the amount 10M of device 10 that extends beyond the edges of end effector 4010.

Next, the user folds/wraps device 10 about the end effector 4010 to further reduce the cross-sectional area of the end effector 4010 and device 10, see FIG. 16D. Care must be taken to ensure that no part of the folded device 10 extends above the top 4010T of the end effector 4010, as this would present the risk of damage to the device 10 by the stitching needles.

At this time, the folded device 10 and attachment system 500 are inserted into the introducer 301L as illustrated in FIG. 16E The sealing member 1000, 1000′, 1000′, 1000′″ is secured to the proximal end of the introducer 310L as shown in FIG. 16F and the procedure continues on such as described with regard to FIGS. 14G-14N or FIGS. 15M-15T, for example.

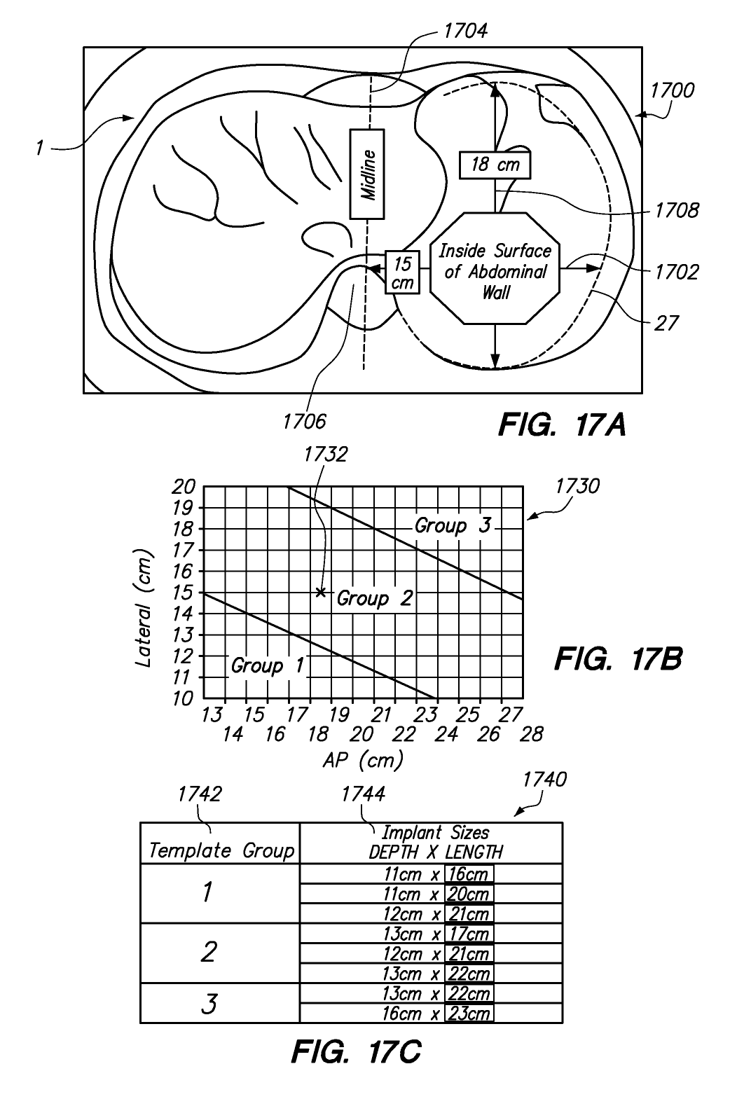

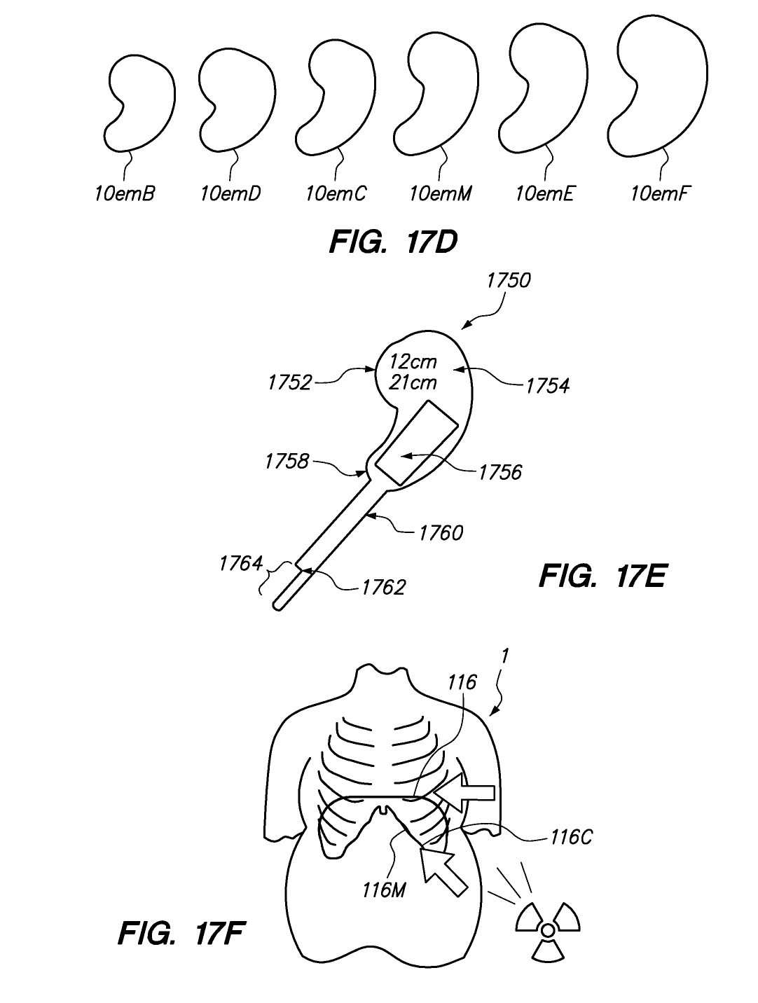

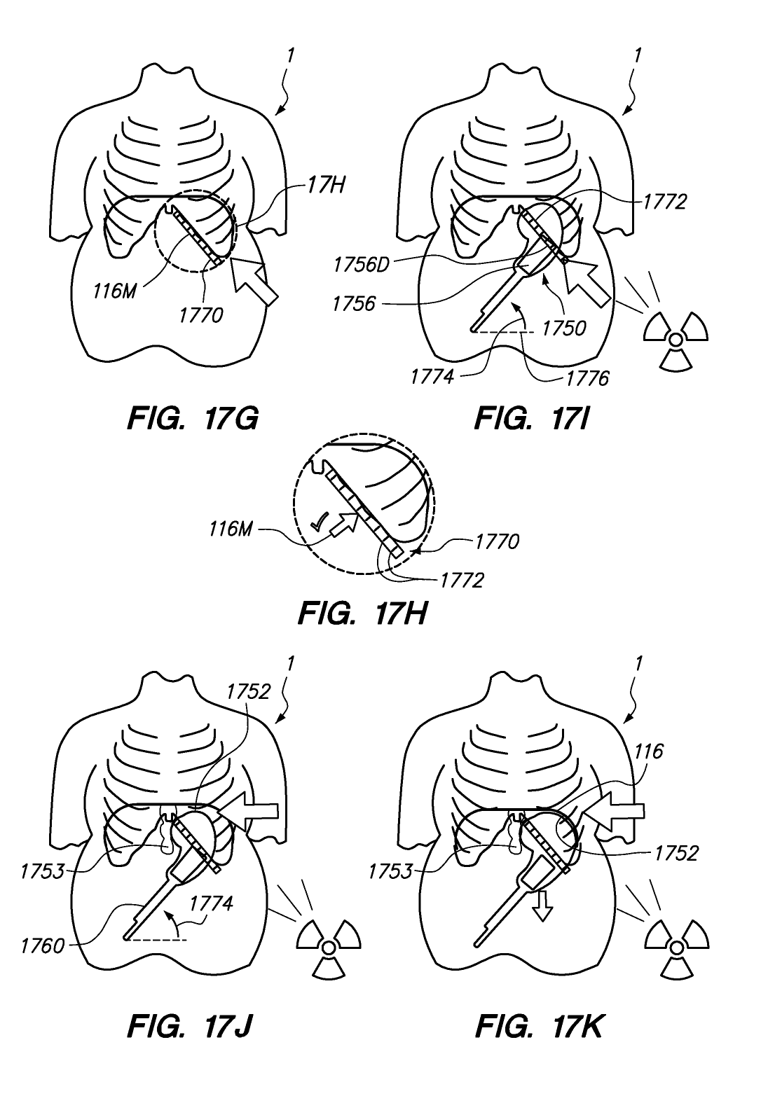

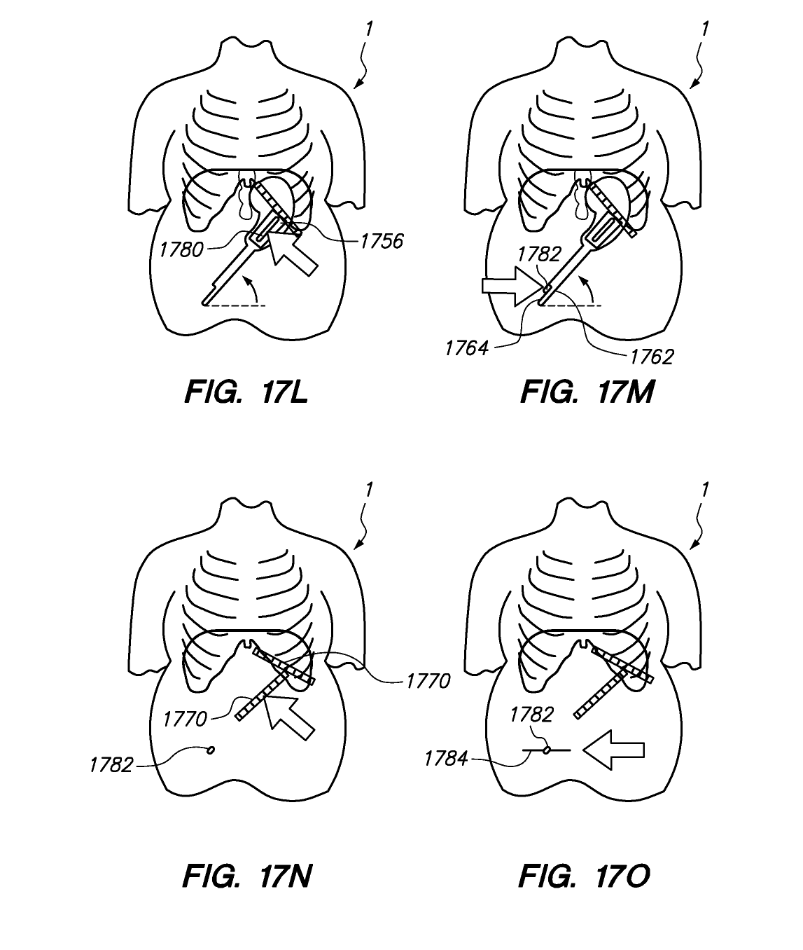

FIGS. 17A-17O are now referred to for a description of templates, template size selection and device size selection that can be practiced according to an embodiment of the present invention. Templates and procedures described can be used in combination with the procedures described in FIGS. 14A-14N or FIGS. 15A-15T, as well as any other implantation procedures described in any of the references that have been incorporated herein that do not conflict with this procedure as described.

FIG. 17A illustrates an abdominal magnetic resonance imaging (MRI) scan 1700 (cross-section) of a patient 1 to be treated by implantation of device 10. The cross-section 1700 is an axial view at the level of the gastroesophageal (GE) junction of the patient 1. A lateral measurement 1702 is made from the midline 1702 to the inside surface of the abdominal wall 27 at a height of the top of the spinal column 1706 in a direction perpendicular to the midline 1702. An AP (anterior to posterior) measurement 1708 is made from the posterior inside rib cage to the anterior inside rib cage perpendicular to the lateral measurement line 1702, midway of the midline 1704 and interior surface of the abdominal wall 27.

Referring to the sizing chart 1730 shown in FIG. 17B, the measurements are plotted on the chart 1730 to determine the appropriate size of the template(s) to be selected for the patient's 1 implantation procedure. In the example shown in FIG. 17A, the Lateral measurement 1702 is about 15 cm and the AP measurement 1708 is about 18 cm. By plotting these values on the chart 1730, as shown by 1732 (or simply looking up the values, using the chart 1730 as a look up chart), the user notes that these measurements indicate the selection of template group 2. If the plotted measurement values fall on a line between two template groupings, the larger template grouping is selected. FIG. 17C is a correlation chart 1740 that correlates Template Group 1742 to implant size 1744, showing various sizes of device 10 and which ones correlate to which template group size.

FIGS. 17D illustrates various sizes of enlargeable members 10emB, 10emD, 10emC, 10emM, 10emE and 10emF from which various sized devices can be constructed. The minimum fill volumes for the enlargeable members are about 790 cc for 10emB, about 950 cc for 10emC, about 1,200cc for 10emM, about 1,440 cc for 10emE and about 2200 cc for 10emF. The approximate depth and length dimensions of the enlargeable members in their nominal (mandrel sized) configurations are as follows: size B: about 11 cm×about 16 cm; size D: about 13 cm×about 17 cm; size C: about 11 cm×about 20 cm; size M: about 12 cm×about 21 cm; size E: about 13 cm×about 22 cm; and size F: about 16 cm×about 23 cm. The appropriate size of enlargeable member to be used is selected by taking the lateral and AP measurements of the patient 1 as described above, referencing the chart in FIG. 17B to identify where the lateral and AP measurements intersect on the chart to identify which of Groups 1-3 is to be used. Next, using the chart in FIG. 17C, the various templates that are included in the Group that was identified are physically overlaid on the patient 1 to determine which one appears to fit the best, with the aid of fluoroscopic landmarks that are compared against the template when it is overlaid. The template 1750 that appears to fit the best is then referenced to identify the depth and length dimensions 1754, 1752 that are marked upon it. These dimensions match one of the enlargeable member sizes, and the size that matches is the size that is selected for use. Alternatively or additionally of the marking of the depth and length dimensions 1754, 1752 on the template 1750, the enlargeable member size (e.g., B,D,C,M,E or F) may be printed otherwise marked on the template 1750. Further alternatively or additionally, the templates 1750 may be color coded to match with corresponding sizes of enlargeable member that can be correspondingly color-coded.

After or before selecting the appropriate size template 1750 and device 10, the patient 1 is prepared for surgery, which may be according to standard laparoscopy protocol, for example. The patient 1 may be positioned supine on the procedure table with the arm on the side of the fluoroscope tucked. Mild reverse Trendlenberg positioning may help placement after sizing of the device. FIG. 17E is an illustration of a positioning template 1750 that is useable to help determine device 10 sizing and placement. The head 1752 of the template 1750 indicates the distal end of the device 10, and a size indication 1754 may be included on the head 1752 to indicate the device 10 size that the selected template 1750 facilitates positioning of. An attachment area cutout is provided for marking the patient in a manner described below. The tail 1758 of the template 1750 indicates the proximal end of the device 10. Handle 1760 indicates the trajectory that the attachment system 500 will take during the implantation procedure. Notch 1762 is used to indicate the proximal-most incision location, and the length and position of tail 1764 indicates the range for the incision location.

Referring to FIG. 17F, the left (patient's left) hemi-diaphragm 116 of the patient is located using visualization equipment such as fluoroscopy (e.g., C-arm fluoroscopy). The left hemi-diaphragm 116 is marked at end inspiration, on the outside of the patient 1, using a surgical marker. The surgeon may palpate the patient's let lower costal margin 116c and mark 116M the margin's inferior edge, on the outside of the patient 1, with a surgical marker, as illustrated in FIG. 17F. Next, a radiopaque, adhesive marker or marking guide 1770, such as a radiopaque adhesive ruler is adhered over the top of the costal margin mark 116M as illustrated in FIGS. 17G-17H. The tic marks 1772 of the marker 1770 are aligned with the inferior edge of the costal margin 116c.

In FIG. 17I, a template size (having the same dimensions as one of the “Implant Sizes” 1744 in FIG. 17C) is selected from group of implant sizes 1744 corresponding to the template group 1742 number (FIG. 17C) that was determined using the anatomical measuring described with regard to FIG. 17A above and use of chart 1730 described above with reference to FIG. 17B. It is noted that eight implant size entries are made in 1744 because two of the sizes appear in two different template groups 1742 because those sizes fall on borders between two Groups. Following the example described with regard to FIGS. 17A-17C, a 12 cm×21 cm template was selected as illustrated in FIG. 17E.

In FIG. 17I, the template 1750 is placed on the patient 1 with the distal edge 1756D of the cutout 1756 adjacent to the inferior edge of the tic marks 1772, and with the handle 1760 oriented at a predetermined angle 1774 to the imaginary horizontal line 1776 that is substantially perpendicular to the midline or spinal column. Predetermined angle 1774 is preferably about forty-five degrees, but the present invention is not limited to this specific angle as the predetermined angle could be within a range of about forty to fifty degrees. The described placement is designed to prevent the stitching needles of the assembly 500 from overlapping with the costal margin 116c during attachment of the device 10. The template 1750 should be placed as close as possible to the xiphoid process without violating the aforementioned placement parameters. It is preferable that the head 1752 of the template 1750 does not intersect (overlie) any portion of the spine 1753, as internal body structures located near midline in the patient 1 may displace the device 10 out of its intended position if the intended position indicated by template 150 intersects the spine 1753. Non-intersecting placement of the head 1752 can be confirmed by fluoroscopic visualization, as illustrated in FIG. 17J. If it is not possible not to place the head 1752 so that it does not intersect 1753, then the amount of intersection should be minimized. Desired positioning may require rotating the template 1750 slightly, or moving the template 1750 slightly inferiorly and laterally, or along the edge of the costal margin 116c. The handle 1760 may cross the midline/intersect the spine 1753, as also shown in FIG. 17J.

In FIG. 17K, visualization such as fluoroscopy is used to determine whether the template head 1752 reaches the diaphragm 116, preferably also without intersecting the spine 1753. FIG. 17K shows proper sizing and orientation/placement of template 1750. If, on the other hand, it is observed that the head 1752 of the template 1750 extends above the diaphragm 116, then the user adjusts the placement by moving the template 1750 inferiorly until the head 1752 is adjacent to the diaphragm, but does not extend superiorly of it. This would also result in the cutout area 1756 and distal edge 1756D of the cutout area being located somewhat below the costal margin 116c and tic marks 1772. If this inferior adjustment results in the edge 1756D being placed more than or equal to a predetermined distance (typically about 4 cm, although this may vary) below the costal margin, then a shorter template 1750 should be selected and the procedure described with regard to FIGS. 17I-17K should be repeated. The selection and repetition of the procedure should be iterated until a satisfactory lit of the template 1750 to the patient 1 has been achieved, wherein a satisfactory fit satisfies the requirements described with regard to FIGS. 17I-17K.

If the visualization performed in FIG. 17K reveals that head 1752 (superior end) is significantly below (e.g., about four centimeters or more), the diaphragm 116, then a longer template 1750 is selected and the procedure described with regard to FIGS. 17I-17K is be repeated. The selection and repetition of the procedure should be iterated until a satisfactory fit of the template 1750 to the patient 1 has been achieved, wherein a satisfactory fit satisfies the requirements described with regard to FIGS. 17I-17K.

Care should be taken not to oversize the device 10 used for implantation into the patient, as this may cause malpositioning of the device 10 when it settles into place after attachment to the patient 1. Once the size of the device 10 to be used has been determined and the location for placement and attachment of the device 10 has been determined, a surgical marking pen can be used to mark the device attachment area on the skin of the patient 1 by drawing a line lengthwise in the center of cutout area 1756 as shown in FIG. 17L. Additionally, the surgical marking pen can be used to make a mark 1782 on the skin of the patient at the notch 1762 (FIG. 17M) or at a location proximal (inferior) to the notch 1762 along tail 1764. The mark should not be made closer to the costal margin 116c than the distance separating the notch 1762 and the costal margin 116c, as this may not allow the attachment system 500 to function properly.

After accomplishing the markings 1780 and 1782, the template 1750 is removed and another adhesive marker 1770 can be adhered to the skin of the patient overlying mark 1780, as shown in FIG. 17N. To identify the location of the incision to be made, a line is drawn (such as with a surgical marker) wherein the line is centered at the incision mark 1782, see FIG. 17O. Optionally, local anesthetic may be applied to the skin over the location of the incision to be made. An incision is next made and the procedure continues as described with regard to FIGS. 14A-14N or FIGS. 15A and 15D-15T, for example. The marker 1770 at the costal margin 116c helps the surgeon to visually identify when the tip of the dilator 570 (FIGS. 14E and 15K) is at (or slightly above) the costal margin 116c. Direct visualization using endoscope 330 can also be used to assist in the visualization. The marker 1770 over line 1780 and line 1780 are used to visualize alignment with the cannula 310L to ensure that cannula 310L maintains alignment with line 1780 at all times.

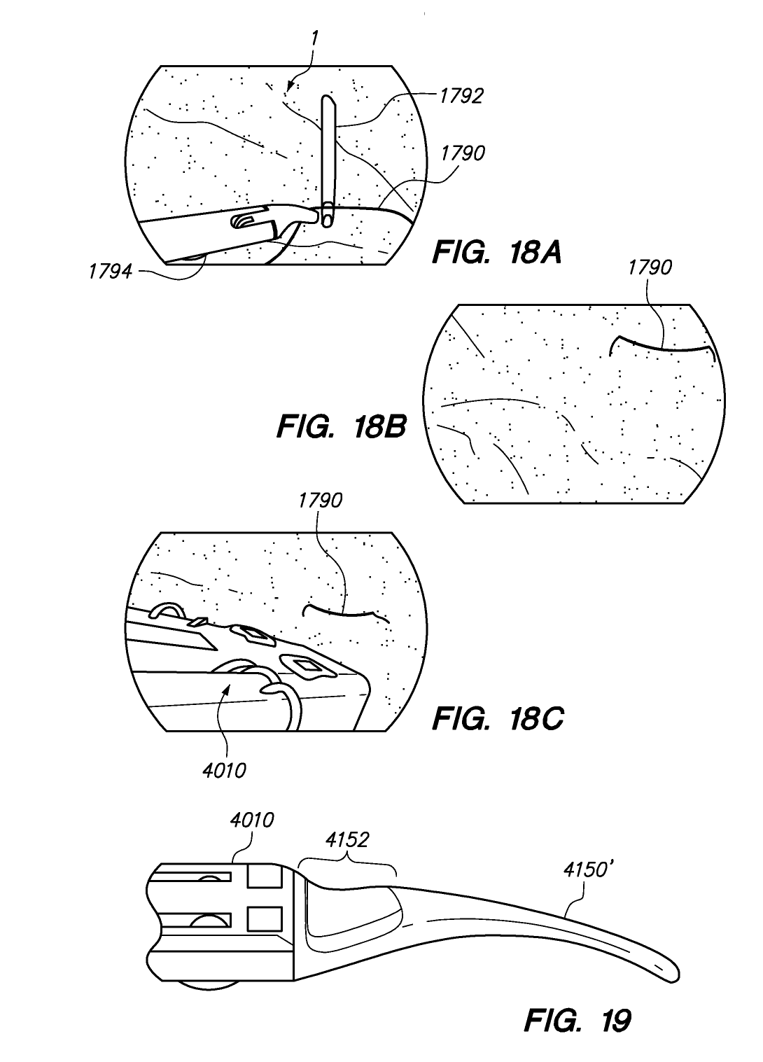

Alternative or in addition to the placement of mark 116M and/or marker 1770 over the costal margin, a suture marker 1790 may be placed as illustrated in FIGS. 18A-18C. A suture is placed as suture marker 1790, for example, using laparoscopic techniques. A suture passer instrument 1792 is used to puncture the skin and abdominal wall of the patient 1 at the inferior edge of costal margin 116c, with the abdomen of the patient 1 under insufflation, as illustrated in FIG. 18A. Another instrument, such as graspers 1794 are inserted through a laparoscopic port and the working end of the instrument 1794 is operated to grasp the suture 1790. The instrument 1794 is next used to draw the suture 1790 laterally (or medially, depending upon which side of the mark that the suture passer 1792 entered on) along the inferior edge of the costal margin 116c. The suture passer 1792 is then reinserted at the opposite end of the mark to engage the suture 1790 and draw it back out of the patient's abdomen, leaving a portion of the suture extending between the entry and exit locations to form the suture marker 1790 as illustrated in FIG. 18B. Thus the suture marker is located along the inferior edge of the costal margin 116c when viewed by fluoroscopy. The suture marker 1790 is typically placed to have a length that is approximately equal to the width of the working end 4010 of attachment tool 4000. The suture marker 1790 marks the distal edge of the position (landing zone) where the attachment tab 150 will be attached. When the device 10 and assembly 500 are introduced into the abdominal cavity, the distal end of 4010 can be positioned adjacent marker 1790 as shown in FIG. 18C.

FIG. 19 is a partial view showing a distal end portion of stitching instrument 4000 that employs an alternative implant guide 4150′ according to an embodiment of the present invention. Guide 4150′ has a notch, concavity or depression 4152 its proximal end portion, adjacent to the proximal end that joins the distal end of end effector (working end) 4010. Depression 4152 is configured and dimensioned to conform to the lowermost rib of the patient at the costal margin 116c so as to function as a jig to properly distance the end effector 4010 (and stitches subsequently placed thereby) from the costal margin. Thus, as the end effector 4010 approached the costal margin 116c as illustrated in FIG. 18C, the surgeon can direct the distal end of the assembly 500 upward and feel when depression 4152 engages the lower most rib. This, in addition to the visualization techniques already described, further facilitates appropriate placement of the stitches and attachment of the device 10 in the desired location.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, too, instrument, device, process, process step or steps, to the objective, spirit and scope of the present invention. All such modifications are intended to be within the scope of the claims appended hereto.

That which is claimed is:

1. A method comprising:
selecting a template from a plurality of different sizes of templates based on measurements of the abdominal cavity of a patient;
orienting said template on the patient at a location overlying the abdominal cavity to select an appropriate size implant;
marking an incision location and an indicator of an angle of approach; and
removing said template from the patient, wherein marks made by said marking remain on the patient.

2. The method of claim 1, comprising using fluoroscopic imaging to facilitate selection of said appropriate size implant.

3. The method of claim 1, further comprising selecting said appropriate size implant from a plurality of different sizes of implants.

4. The method of claim 1, wherein said implants are enlargeable implants.

5. The method of claim 1, further comprising:
marking the patient at a location overlying a portion of the costal margin, prior to said orienting, and wherein said orienting includes positioning a superior edge of a cutout in said template adjacent to or inferior of a mark made by said marking of the location overlying a portion of the costal margin.

6. The method of claim 1, further comprising adhering a marking guide to an indicator location as said indicator of the angle of approach.

7. The method of claim 1, wherein said template includes a cutout indicating a location where said implant is to be attached to the abdominal wall, and wherein said indicator of the angle of approach comprises a mark drawn within bounds of said cutout.

8. The method of claim 6, further comprising adhering a marking guide to the patient so that said marking guide overlies said mark drawn within bounds of said cutout.

9. The method of claim 1, further comprising positioning said template so that a head of said template approximates the diaphragm of the patient, but does not extend superiorly of the diaphragm.

10. The method of claim 1, wherein said marking the incision location comprises marking adjacent to a notch in a tail of said template or inferior of said notch, adjacent to a portion of said tail inferior of said notch.

11. The method of claim 1, further comprising adhering a marking guide to a location overlying a portion of the costal margin.

12. The method of claim 5, further comprising adhering a marking guide to the patient, wherein said marking guide overlies a mark made by said marking the patient at a location overlying a portion of the costal margin.

13. The method of claim 1, further comprising placing a suture marker that extends along the internal surface of the abdominal wall along the inferior edge of a portion of the costal margin of the patient.

14. The method of claim 4, further comprising:
making an incision or puncture through the patient's skin at said marked incision location;
establishing a delivery tract through an opening formed by said incision or puncture, subcutaneous fat and fascia and into the patient's abdominal cavity, but not through the stomach;
dilating the opening and placing an introducer cannula along the tract such that said introducer cannula extends from a location outside of the patient to a location within the abdominal cavity;
inserting an instrument and said selected enlargeable implant into said introducer cannula, wherein said enlargeable implant is mounted on a distal end portion of said instrument and said enlargeable implant is in a compact configuration;
enlarging said implant to an enlarged configuration;
attaching said implant to an inner surface of the abdominal cavity;
removing said instrument and introducer cannula;
attaching an adjustment member to a fill tube in fluid communication with said implant; and
closing the opening.

15. The method of claim 14, further comprising:
prior to said enlarging said implant to an enlarged configuration, retracting said introducer cannula relative to said instrument and implant to expose said implant and a working end of said instrument out of a distal end of said introducer cannula.

16. The method of claim 14, further comprising:
prior to said attaching said implant, contacting a lowermost rib of the patient with a depression formed in a distal end portion of the instrument extending distally of an end effector of said instrument.

17. The method of claim 14, further comprising:
attaching a sealing member mounted on said instrument to a proximal end of said introducer cannula to seal off said introducer cannula; and
insufflating the abdominal cavity of the patient.

18. The method of claim 14, further comprising:
prior to said attaching said implant, verifying a correct positioning of said implant by verifying alignment of said instrument with said indicator of the angle of approach.

19. The method of claim 14, further comprising:
prior to said attaching said implant, verifying a correct positioning of said implant by verifying alignment of said instrument with at least one of said indicator of the angle of approach and said suture marker.

20. The method of claim 14, further comprising:
prior to said attaching, verifying a position of a working end of said instrument relative to said marking guide that overlies a portion of the costal margin.

21. The method of claim 14, further comprising verifying, using direct laparoscopic visualization, a location of a distal end of an end effector of said instrument relative to the costal margin of the patient.

22. The method of claim 14, wherein said dilating the opening and placing an introducer cannula comprises inserting a distal end of a dilator through the opening, wherein said introducer cannula is mounted over said dilator and a distal end portion of said introducer cannula is passed through the abdominal wall along the tract;
the method further comprising removing said dilator prior to said inserting an instrument and enlargeable implant into said introducer cannula; and insufflating the abdominal cavity.

23. The method of claim 14, wherein the opening is the only opening formed in the patient to carry out the entirety of the method.

24. The method of claim 14, wherein said implant is attached to fascia and/or peritoneum.

25. The method of claim 14, wherein said implant is attached to abdominal muscle.

26. The method of claim 14, wherein said instrument comprises an attachment tool and a suturing tool that are releasably connected to one another, wherein said attaching comprises attaching said implant using said attachment tool, and then disconnecting said attachment tool from said suturing tool and removing said attachment tool from said introducer cannula.

27. The method of claim 26, further comprising tightening the attachment of said implant to the inner surface of the abdominal cavity using said suturing tool; and wherein said removing said instrument comprises removing said suturing tool after completing said tightening said attachment.

28. The method of claim 14, further comprising removing at least a portion of a falciform ligament.

29. An apparatus for use in a minimally-invasive abdominal surgical procedure, said apparatus comprising:
an elongate introducer cannula having a tubular main body, a distal end, a proximal end and a main lumen extending therethrough;
a stitching instrument having at least one elongate shaft insertable through said introducer cannula, said instrument having a length greater than a length of said elongate introducer;
a sealing member forming a seal around said at least one elongate shaft of said instrument and configured to form a seal between said instrument and said introducer cannula to seal off said main lumen; and
an enlargeable implant releasably attached to a distal end portion of said instrument.

30. The apparatus of claim 29, wherein said stitching instrument comprises an attachment tool and a suturing tool that are releasably connected to one another, wherein said attaching comprises an end effector having needles configured to drive stitches to attach said implant to a patient.

31. A sealing member for forming a seal between an introducer cannula and an attachment tool configured to attach an implant in the abdominal cavity, said sealing member comprising:
a main body having a generally circular cross-sectional configuration;
attachment members configured and dimensioned to attach to a proximal end of said introducer cannula;
a sealing ring configured to seal with an opening in fluid communication with a main lumen of said introducer cannula;
an opening configured to allow passage of an end effector having a first cross-sectional area, as well as a shaft having a second cross-sectional area different from said first cross-sectional area; and
a valve formed around the opening and configured to form a seal with said shaft.

32. The sealing member of claim 31, wherein said opening comprises a first opening and said valve comprises a first valve, said sealing member comprising a second opening for receiving a tool or instrument therethrough and forming a seal therewith, and a second sealing member formed around said second opening.

33. An implantable device for treatment of obesity, said device comprising:
an expandable main body member configured to be positioned adjacent a portion of a stomach of a patient, within the abdominal cavity of the patient wherein said expandable main body member comprises a wall surrounding an internal chamber;
an attachment tab interfacing with an outer surface of said wall and extending from said wall of said expandable main body member, said attachment tab configured to fix a portion of said main body member to and in contact with a portion of at least one internal body structure;
an inner backing layer interfacing with an inner surface of said wall and bonded thereto; and
wherein said attachment tab, said wall and said inner backing layer, are bonded together.

34. The device of claim 33, wherein said attachment tab and said inner backing layer are bonded together through at least one opening through said wall and wherein each said opening is sealed by the bonding together of said attachment tab and said inner backing layer.

35. The device of claim 33, further comprising a plug bonded to said attachment tab, said wall and said inner backing layer, said plug having placed in an opening in said wall, said plug having been bonded with said attachment tab, wall and inner backing layer, thereby filling said opening in which said plug was inserted prior to having been bonded.

36. A method of making an implantable device for treatment of obesity, said method comprising:
providing an expandable main body member configured to be positioned adjacent a portion of a stomach of a patient, within the abdominal cavity of the patient wherein said expandable main body member comprises a wall surrounding an internal chamber and an opening through said wall;
laying a layer of an attachment tab on an outer surface of said wall over a location of said opening;
contacting an inner backing layer to an inner surface of said wall under a location of said opening; and
bonding said wall, layer of an attachment tab and inner backing layer together.

37. The method of claim 36, further comprising:
inserting a plug in said opening; and
wherein said bonding comprises bonding said wall, said plug, said layer of an attachment tab and said inner backing layer together.

38. The method of claim 36, wherein said bonding comprises vulcanizing.

39. The method of claim 37, wherein said bonding comprises vulcanizing.
